(19) United States
(12) Patent Application Publication
(10) Pub. No.: US 2009/0281372 A1
(43) Pub. Date: Nov. 12, 2009

(54) CARDIAC HARNESS ASSEMBLY FOR TREATING CONGESTIVE HEART FAILURE AND FOR DEFIBRILLATION AND/OR PACING/SENSING

(75) Inventor: Matthew G. Fishler, Sunnyvale, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: PARACOR MEDICAL, INC., Sunnyvale, CA (US)

(21) Appl. No.: 12/115,704

(22) Filed: May 6, 2008

(51) Int. Cl. A61F 2/00; B29C 45/14

(52) U.S. Cl. 600/37; 264/261

(57) ABSTRACT

A cardiac harness assembly for treating congestive heart failure and for use in defibrillation and/or pacing/sensing is provided. The cardiac harness includes a number of longitudinal ribs spaced apart by connectors, the longitudinal ribs extending from the base to the apex of the heart. The longitudinal ribs have a high degree of longitudinal flexibility so that when the cardiac harness is mounted on the heart, the longitudinal ribs flex along the longitudinal axis of the ribs as the heart expands and contracts circumferentially. The cardiac harness provides a uniform and continuous compressive force on the heart throughout the cardiac cycle.

### BACKGROUND OF THE INVENTION

The present invention relates to a device for treating heart failure. More specifically, the invention relates to a cardiac harness configured to be fit around at least a portion of a patient's heart.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical changes to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus a vicious cycle can result, in which dilation leads to further dilation and greater functional impairment.

Historically, congestive heart failure has been managed with a variety of drugs. Devices have also been used to improve cardiac output. For example, left ventricular assist pumps help the heart to pump blood. Multi-chamber pacing has also been employed to optimally synchronize the beating of the heart chambers to improve cardiac output. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart.

Although some of the above-discussed devices hold promise, there remains a need in the art for a device for treating CHF to prevent a remodeled heart from further remodeling and/or help reverse remodeling of a diseased heart.

### SUMMARY OF THE INVENTION

The present invention relates to a device for treating heart failure. More specifically, the invention relates to a cardiac harness configured to fit around at least a portion of a patient's heart. The cardiac harness may include electrodes attached to a power source for use in defibrillation and/or pacing/sensing. The present invention cardiac harness can be used to treat congestive heart failure and to reverse remodel a heart that has grown in size as a result of congestive heart failure. Further, the harness may prevent dilation of the heart from occurring from initiation or progression of heart failure.

The present invention relates to a cardiac harness for treating congestive heart failure and other diseases. The cardiac harness includes a plurality of longitudinal ribs that are spaced apart and each having a base end and an apex end. The base end of the longitudinal ribs corresponds to the base of the heart, while the apex end of the longitudinal ribs corresponds to the apex of the heart. Multiple connectors attach adjacent longitudinal ribs to each other, so that the cardiac harness resembles a number of ladders connected together with the rungs of the ladders being the connectors between the adjacent longitudinal ribs. In one embodiment, the connectors extend only between adjacent ribs, while in other embodiments the connectors can extend and attach to multiple ribs. The longitudinal ribs have a high degree of longitudinal flexibility so that when the cardiac harness is mounted on the heart, the longitudinal ribs can flex circumferentially along the longitudinal axis of the ribs as the heart expands and contracts circumferentially throughout the cardiac cycle. In one embodiment, the longitudinal ribs are made out of a superelastic alloy, such as nitinol, while the connectors are made out of a polymer material, such as silicone rubber.

The cardiac harness of the present invention can be used to treat congestive heart failure, or other heart diseases, by providing a cardiac harness having a plurality of longitudinal ribs spaced apart and a plurality of connectors attaching adjacent ribs together. The cardiac harness has an at-rest configuration and a first circumference C₁, and an expanded configuration when mounted on the heart at end diastolic filling. The expanded configuration defines a second circumference C₂. When the cardiac harness is mounted on the heart, C₁ is substantially less than C₂. The longitudinal ribs elastically deform with a spring-like force as the cardiac harness expands from the C₁ configuration to the C₂ configuration.

The cardiac harness of the present invention can be mounted on the heart by minimally invasive means. While it is possible to mount the cardiac harness on the heart through open heart surgery (via a median sternotomy), the preferable method is by minimally invasive surgery. In one embodiment, the cardiac harness has a plurality of longitudinally flexible ribs spaced apart and a plurality of connectors attaching adjacent ribs together. The cardiac harness is compressed into a tubular housing that has a diameter that is sufficiently small so that the housing can be inserted through a minimally invasive opening between a patient's ribs. The tubular housing is advanced through the minimally invasive access site in the patient so that the tubular housing is positioned proximate the apex of the heart. The cardiac harness is then advanced out of the tubular housing and over the apex of the heart. The cardiac harness is further advanced onto the heart so that it is mounted on the heart and covers a substantial portion of the heart. The flexible longitudinal ribs of the cardiac harness provide column strength as the cardiac harness is pushed out of the tubular housing and advanced onto the heart.

The cardiac harness of the present invention can be made by imparting a predetermined at-rest shape to a plurality of superelastic longitudinal ribs, whereby the ribs can be formed of nitinol. The longitudinal ribs preferably are electropolished and cut to a predetermined length. In one embodiment, the longitudinal ribs, in an at-rest pattern, are positioned between two sheets of silicone rubber, and then at least one of the sheets is vulcanized thereby entrapping or encasing the longitudinal ribs in the silicone rubber sheets. Excess silicone rubber can be removed (e.g., by laser cutting) so that a plurality of connectors are formed between adjacent longitudinal ribs, thereby forming the cardiac harness.

In another embodiment for making the cardiac harness, a mold is provided for receiving a plurality of longitudinally spaced ribs. The longitudinal ribs are placed in the mold so that the ribs are substantially parallel to each other. The mold has a plurality of channels between the ribs, whereby the channels at the base end of the mold have a curve with a greater amplitude (longer path length) relative to the channels moving toward the apex portion of the mold which have a curve with a progressively smaller amplitude (shorter path length). An elastomer is injection molded into the mold so that the ribs are encased in the elastomer and the connectors are formed in the channels, thereby connecting adjacent ribs together. In one embodiment, the elastomer is silicone rubber. Alternatively, the ribs are jacketed in extruded silicone rubber tubing prior to placing the ribs in the mold. When the cardiac harness is removed from the mold, the curves in the connectors will straighten so that the longitudinal ribs of the cardiac harness remain connected to each other, however, the ribs are no longer parallel since the amplitude of the curve at the base of the cardiac harness is greater than the amplitude of the curves progressively moving toward the apex portion of the cardiac harness. Thus, the longitudinal ribs form a tapered configuration whereby there is a greater spacing between the base end of the longitudinal ribs and a smaller spacing between the longitudinal ribs moving toward the apex end of the longitudinal ribs.

Further features and advantages of the present invention will become apparent to one of skill in the art in view of the detailed description of the preferred embodiments which follows, when considered together with the attached drawings and claims.

### BRIEF DESCRIPTION OF THE DRAWINGS

FIG. 1 is an elevational view depicting one embodiment of a cardiac harness having longitudinal ribs.

FIG. 2 is an elevational view depicting the cardiac harness of FIG. 1 mounted on an artificial heart.

FIG. 3A is a side view depicting a connector that attaches longitudinal ribs to each other.

FIGS. 3B-3F are cross-sectional views depicting different embodiments of the connector of FIG. 3A.

FIGS. 4A-4E are front views of different embodiments of connectors for attaching the longitudinal ribs to each other.

FIG. 5A is a front view of a longitudinal rib.

FIGS. 5B-5H are transverse cross-sectional views depicting different embodiments of the longitudinal rib of FIG. 5A.

FIGS. 6A and 6B are plan views of a portion of cardiac harness depicting the longitudinal ribs in an at-rest configuration.

FIGS. 7A-7B are plan views depicting portions of the cardiac harness of FIGS. 6A and 6B respectively where the longitudinal ribs are in an expanded configuration.

FIGS. 7C-7K are plan views depicting portions of a cardiac harness with different embodiments of connectors between adjacent ribs.

FIGS. 8A-8D are plan views depicting a portion of the end of a longitudinal rib.

FIG. 9 is a plan view depicting a portion of a cardiac harness where the longitudinal ribs are attached by a band at the base end and the apical end of the cardiac harness.

FIGS. 10A-10B are plan views depicting two different embodiments of longitudinal ribs.

FIG. 11 is a perspective view depicting a tubular housing for receiving the cardiac harness.

FIG. 12 is an end view depicting a tubular housing with the cardiac harness compressed within the housing.

FIG. 13 is a plan view depicting the cardiac harness being advanced out of the tubular housing.

FIG. 14 is a plan view depicting the cardiac harness further advancing out of the tubular housing.

FIG. 15 is a plan view depicting a portion of the tubular housing and the cardiac harness being fully advanced out of the housing and in an expanded configuration.

FIG. 16 is a plan view of a prototype cardiac harness in an expanded configuration.

FIG. 17 is a partial plan view of the prototype cardiac harness of FIG. 16 in an expanded configuration.

FIG. 18 is a plan view depicting a prototype cardiac harness in a compressed configuration.

FIG. 19 is a partial plan view of the prototype cardiac harness of FIG. 18 in a compressed configuration.

FIG. 20A is a plan view of a cardiac harness depicting connectors having varying amplitudes.

FIG. 20B is a plan view of the cardiac harness of FIG. 20A in which the curved connectors have been straightened to form a cardiac harness having a tapered configuration.

FIG. 21 is a partial plan view depicting a portion of a cardiac harness having a pace/sense electrode positioned mid-wall between longitudinal ribs.

FIG. 22 is a partial elevational view depicting a portion of a cardiac harness showing a pace/sense electrode positioned between longitudinal ribs near the base of the harness.

FIGS. 23-24 are side views of a pace/sense electrode having a first position in one embodiment, and being relocated to a second position in a second embodiment.

FIGS. 25 and 26 are top and front views respectively depicting a bipolar pace/sense electrode with a flattened distal section.

FIG. 27 is a partial plan view depicting a portion of a cardiac harness where a pace-sense electrode is partially advanced into a guide-sheath.

FIG. 28 is a partial plan view depicting a portion of a cardiac harness where a pace-sense electrode has been advanced toward the apical of a guide-sheath.

FIG. 29 is a partial plan view depicting a portion of a cardiac harness with a pace/sense electrode fully advanced and inserted within a base portion of a guide-sheath.

FIG. 30 is a partial plan view depicting a portion of a cardiac harness having a guide-sheath molded into and in line with the longitudinal ribs of the cardiac harness and depicting a defibrillation electrode and lead inserted into the sheath.

FIG. 31 is a partial plan view depicting a portion of a cardiac harness having a guide sheath molded into and aligned with the longitudinal ribs of the harness.

FIG. 32 is a partial plan view depicting a portion of a cardiac harness with a guide-sheath attached to the pericardial side of the connector.

FIG. 33 is a partial plan view depicting a portion of a cardiac harness where a defibrillation electrode is inserted into a guide-sheath.

FIG. 34 is a partial plan view depicting a portion of a cardiac harness where a defibrillation electrode is inserted into the guide-sheath of FIG. 32.

FIG. 35 is a plan view depicting a mold for making a portion of a cardiac harness.

### DETAILED DESCRIPTION OF THE PREFERRED EMBODIMENTS

The present invention cardiac harness differs from prior art harnesses in that it has multiple flexible longitudinal ribs that are spaced apart and that can flex circumferentially and with the expansion and contraction of the heart on which the harness is mounted. The longitudinal ribs are spaced apart by connectors that limit the circumferential expansion of the longitudinal ribs but do not limit the expansion of the heart during end diastole.

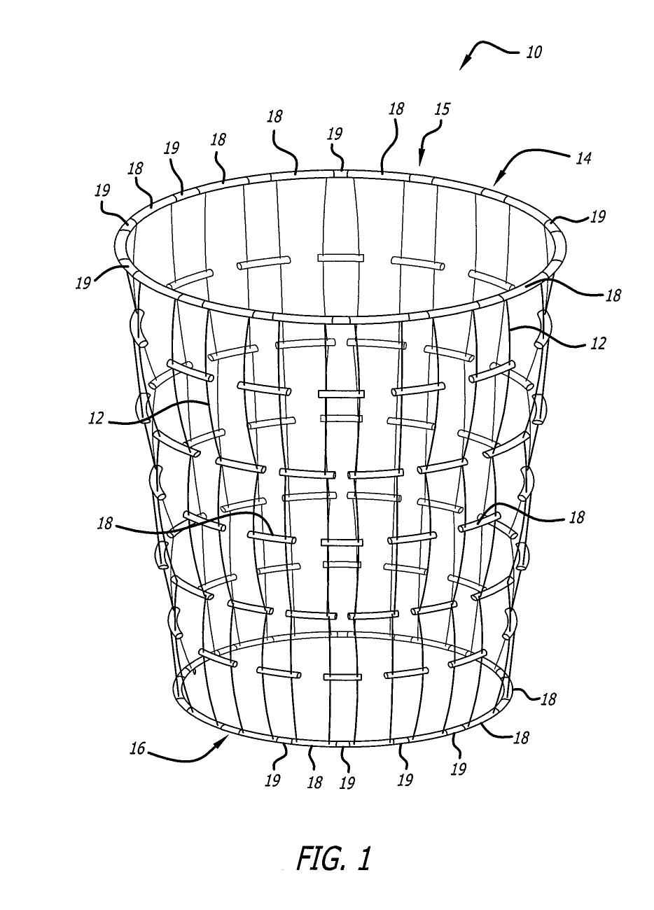

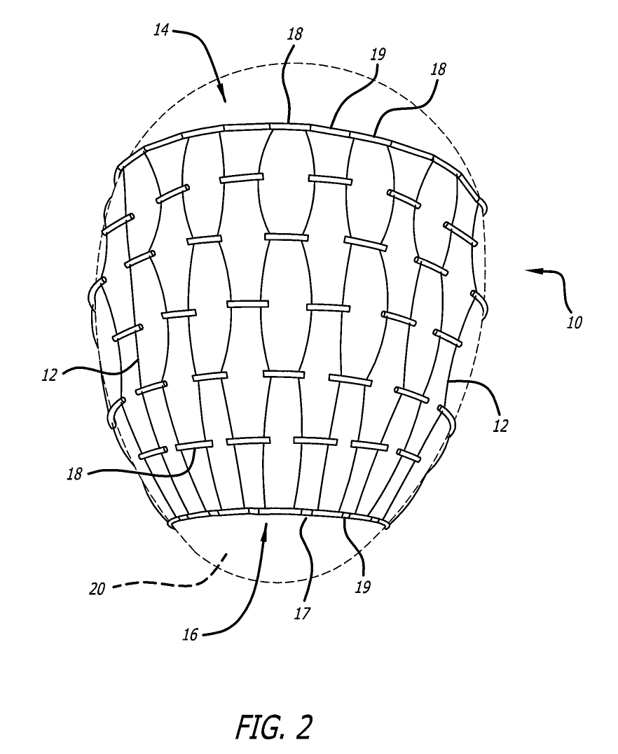

In keeping with the invention, FIGS. 1 and 2 illustrate a cardiac harness 10 having a plurality of longitudinal ribs 12 spaced apart. The cardiac harness has a base end 14 which coincides with the base of the heart and an apical end 16 which coincides with the apex of the heart. The longitudinal ribs are spaced apart and attached to each other by connectors 18 which, in one embodiment, are flexible, however, the connectors 18 preferably do not stretch. In FIGS. 1 and 2, a base band 15 provides attachment points 19 for one end of the longitudinal ribs 12, while an apical band 17 provides attachment points 19 for the apical end of the longitudinal ribs 12. The attachment points can comprise alternating compliant and non-compliant sections 19 positioned between the connectors 18 in order to balance the compliance of the harness 10 with the compliance of the base end 14 and the apical end 16. In one embodiment, the base band 15 and the apical band 17 are flexible and elastic so that as the cardiac harness 10 expands and contracts circumferentially with the beating heart, the base band and the apical band will stretch and contract so that expansion of the heart during end diastole is not limited and the compliance of the cardiac harness 10 (including the base band 15 and apical band 17) is uniform.

Still referring to FIGS. 1 and 2, in one embodiment the longitudinal ribs 12 consist of thirty-six nitinol wires having a diameter of 0.1524 mm (0.006 inch) each, and which are unpolished and uninsulated. Preferably, the nitinol wire longitudinal ribs 12 are all polished and insulated with a dielectric material such as silicone rubber. Adjacent ribs are interconnected by the connectors 18 which consist of silicone rubber having an outer diameter of approximately 0.9398 mm (0.037 inch). The length of the connectors 18 are greater at the base end 14 of the cardiac harness 10 than at the apical end 16 of the cardiac harness so that the longitudinal ribs 12 taper toward the apex of the heart. As an example, the length of the connectors 18 near the base end 14 of the cardiac harness 12 are approximately 5 mm (0.19685 inch), while the connectors 18 near the apical end 16 are approximately 3 mm (0.1181 inch) in length. In one embodiment, the connectors 18 are spaced apart at approximately 19.05 mm (0.75 inch) intervals along the longitudinal dimension of each longitudinal rib 12. In another embodiment, the connectors 18 are spaced approximately at 1 cm (0.3937 inch) intervals along the longitudinal dimension of each longitudinal rib 12. The number of longitudinal ribs 12 and the number and spacing of connectors 18 can be varied to suit a particular purpose. For example, a harness having a greater number of longitudinal ribs than a harness with fewer ribs would be less stiff and provide less compressive force on the heart, all other factors being equal. Further, the diameters of the ribs and connectors can vary, the smaller diameters being more flexible than the relatively larger diameters.

In an alternative embodiment to that shown in FIGS. 1 and 2, relatively short sections of silicone tubing are positioned between the connectors 18 at the base end 14 and the apical end 16 of the cardiac harness 10 in order to provide a complete elastic cardiac support at the ends of the cardiac harness.

In the embodiments disclosed in FIGS. 1 and 2, the cardiac harness 10 is in an at-rest condition in FIG. 1, and a stretched condition in FIG. 2. In one embodiment, the cardiac harness preferably will stretch between 25% and 200% (note that the harness can stretch to over 200%) in the circumferential direction in expanding from the at-rest condition in FIG. 1 to the expanded and stretched condition in FIG. 2. At no time, however, does the cardiac harness limit the expansion of the heart during diastole. In other words, the harness of the present invention will not limit or stop the heart from further expansion during diastole like some prior art jackets do.

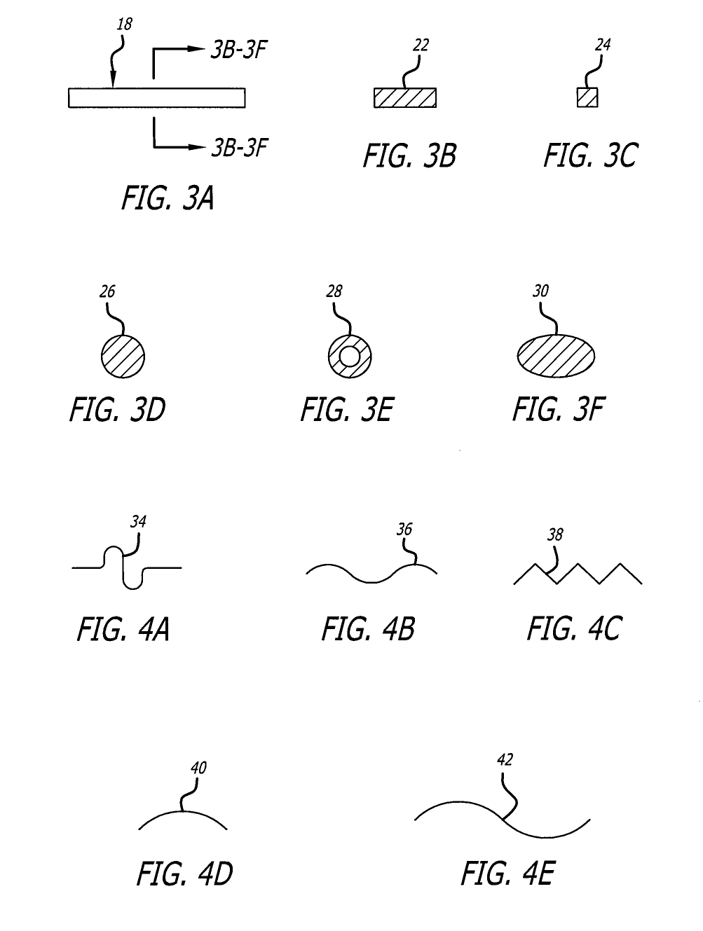

The connectors 18 of the present invention can have virtually any transverse cross-sectional shape as shown in FIGS. 3A-3F. For example, the connectors 18 can have a transverse cross-sectional shape including a rectangular shape 22, a square shape 24, a cylindrical shape 26, a round/hollow or tubular shape 28, or an elliptical/oval shape 30. The choice of which transverse cross-sectional shape is most appropriate can depend upon a number of factors including availability of materials, manufacturability, and ease of connecting the connectors to the longitudinal ribs 12.

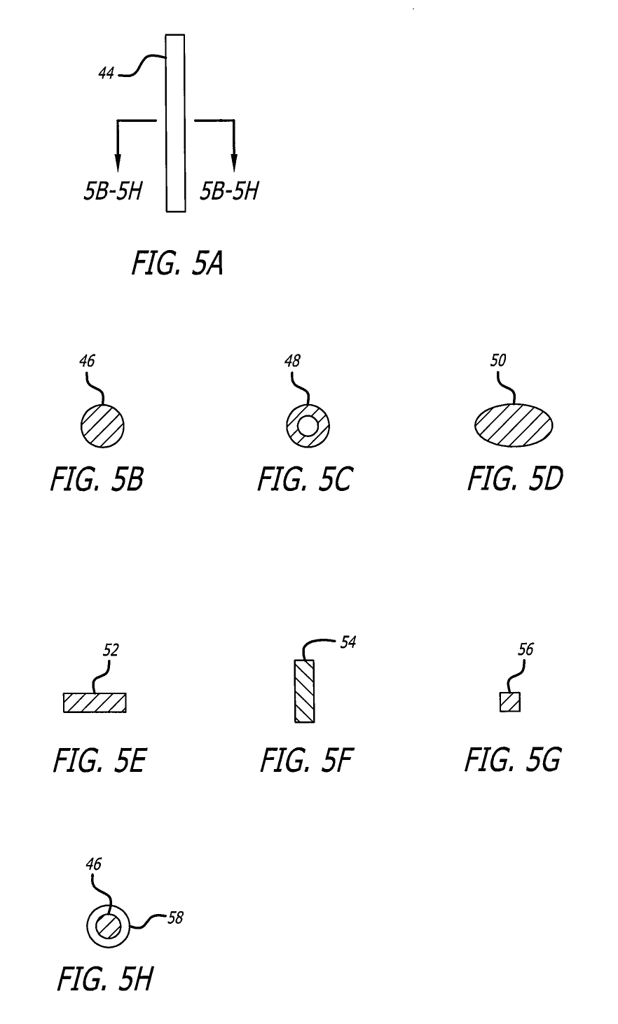

Turning to FIGS. 4A-4E, the connectors 18 can have any of numerous configurations such as a straight connector shown in FIG. 1. The connectors can have a straight configuration with bends 34, an undulating shape 36, a zig zag shape 38, a curved shape 40, or a sinusoidal shape 42 (a bifurcated shape is contemplated but not shown). A straight connector provides little or no circumferential stretch, however, the shapes illustrated in FIGS. 4A-4E can provide some circumferential stretch or expansion depending upon the need.

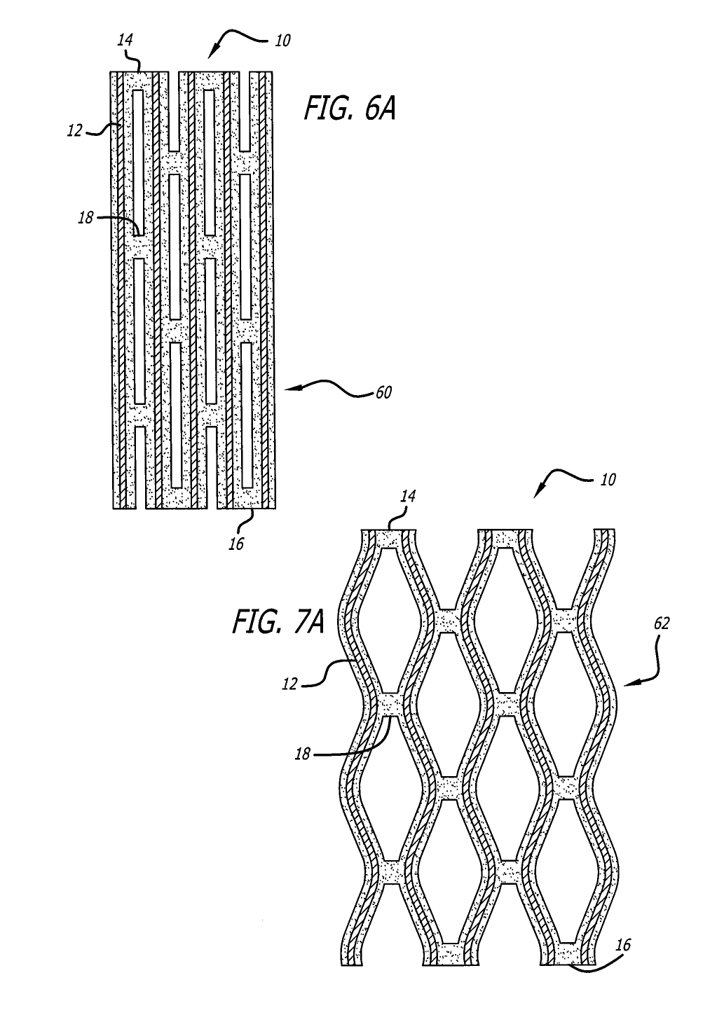

In further keeping with the invention, and referring to FIGS. 5A-5H, the longitudinal ribs can take on a number of configurations. For example, the rib can be a straight rib 44 or it can be two or more ribs in parallel (not shown) that are closely joined together. As shown in FIG. 5A, the rib is a substantially straight rib 44 and can have a number of cross-sectional shapes as shown in FIGS. 5B-5H. Among the transverse cross-sectional shapes for the straight rib 44, are a circular rib 46, a tubular rib 48, an elliptical-shaped rib 50, a horizontal rectangular-shaped rib 52, a vertical rectangular-shaped rib 54, a square-shaped rib 56, and a solid core circular rib 46 with a coating 58 such as silicone rubber. Preferably, all of the differently configured longitudinal ribs 44 will be electrically insulated with any known dielectric material such as silicone rubber, polyurethane, parylene, and the like. Each of the enumerated transverse cross-sectional shapes of the longitudinal ribs 12 will provide different flexibilities in the longitudinal and circumferential direction. As an example, the cylindrical-shaped rib shown in FIG. 5B should have a longitudinal flexibility that is uniform in all directions. In contrast, with reference to the elliptically or oval-shaped cross-sectional-shaped rib 50 of FIG. 5D, the rib will be longitudinally less flexible along the major axis of the ellipse, and more longitudinally flexible along the minor axis of the ellipse. Thus, depending upon the application, different transverse cross-sectional shapes can be used to deliver different flexibilities for the longitudinal ribs 12.

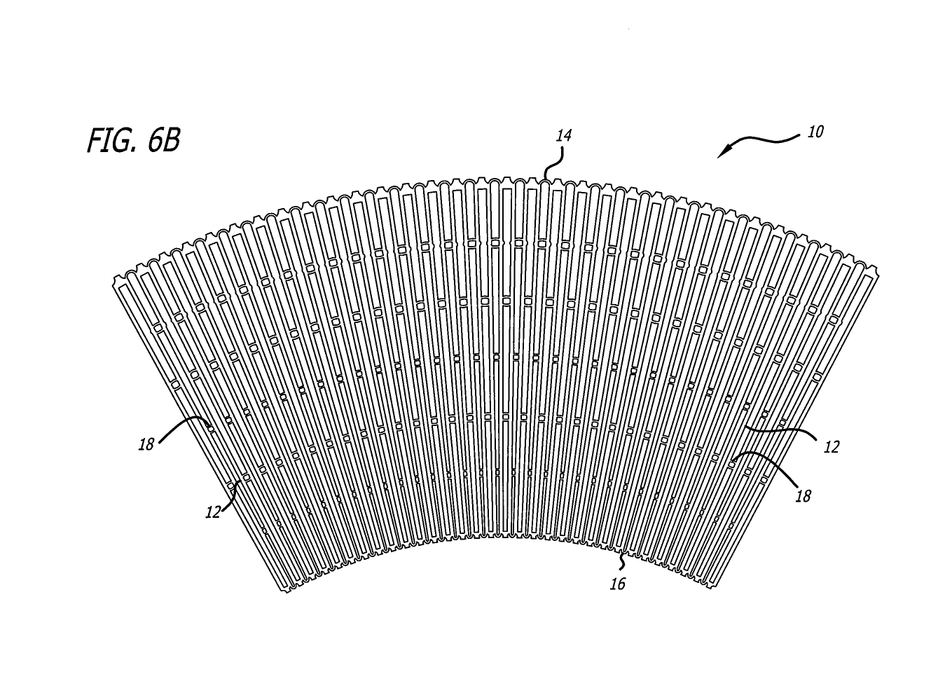

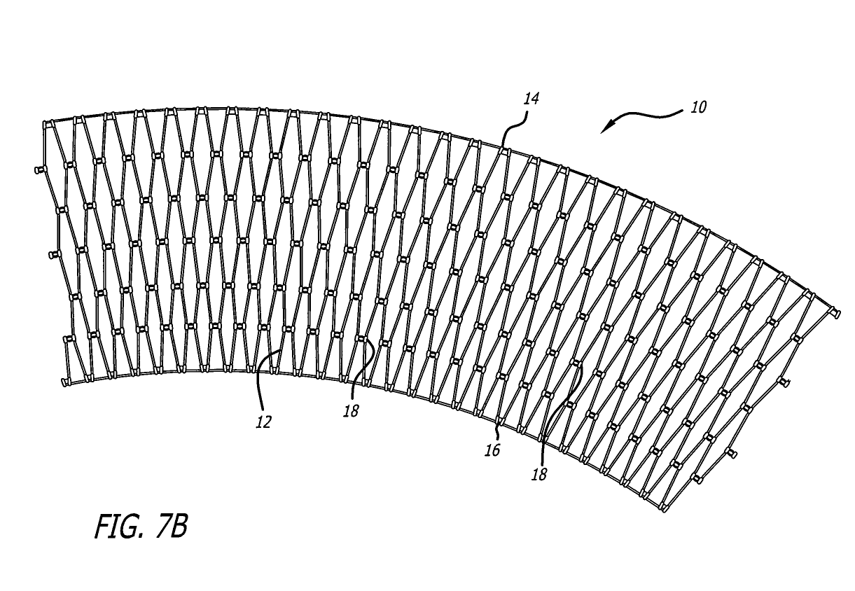

In further keeping with the invention, a section of the cardiac harness 10 is shown in an at-rest configuration 60 in FIG. 6A, and an expanded configuration 62 in FIG. 7A. In this embodiment, the base end 14 and the apical end 16 form the longitudinal extremities of the harness, and the connectors 18 extend between longitudinal ribs 12. In this embodiment, the longitudinal ribs are coated with a coating such as a silicone rubber coating 66 which also forms the connectors 18. Referring to FIG. 7A, cardiac harness 10 is shown in its expanded configuration 62 as it would appear if it were mounted on a heart. The longitudinal ribs 12 expand circumferentially unless constrained by the connectors 18 which are substantially non-distendible and attach the adjacent longitudinal ribs. As can be seen in FIG. 7A, connectors 18 are staggered from one pair of adjacent longitudinal ribs to the next, so that the longitudinal ribs can expand circumferentially in a uniform manner as the heart expands and contracts during the regular cardiac cycle. Importantly, the longitudinal ribs 12 are configured to provide a compressive force on the heart at all times, and as the heart expands the compressive force remains elastically compliant in order to relieve wall stress on the heart. By stating that the compressive force remains substantially uniform as the heart expands, it is meant that the cardiac harness is formed from a nitinol alloy that is highly elastic in nature. With respect to one embodiment of the cardiac harness of the present invention, it is formed from nitinol longitudinal ribs which behave in a typical stress/strain relationship as the strain is increased. Thus, if the stress is on the vertical axis of the stress/strain curve, and strain is on the horizontal axis, as the strain increases the stress also increases up to a point where the stress remains elastically compliant or uniform, even as the strain continues to increase. Similarly, as the strain is decreased, the stress travels back down the same curve in an elastic manner. Thus, the nitinol cardiac harness is highly elastic and does not plastically deform even when a high degree of strain is applied. Since the cardiac harness of FIGS. 6A, 6B, 7A and 7B have a silicone rubber coating, the longitudinal ribs 12 are electrically insulated so the harness can be used with other devices such as implantable defibrillators or pacing and sensing devices commonly known in the art.

The cardiac harness 10 of FIGS. 6A and 6B generally are undersized relative to the circumference of the heart upon which the cardiac harness 10 is mounted. For example, in the at-rest configuration as shown in FIGS. 6A and 6B, the cardiac harness can be anywhere from about 25% to up to over 200% by circumference of the circumference of the heart upon which it is going to be mounted. In other words, the at-rest cardiac harness 60 circumference must be expanded from the at-rest circumference to the expanded circumference as shown in FIGS. 7A and 7B in order to be mounted on the heart. Once mounted on the heart, the cardiac harness applies a continuous compressive force on the order of approximately 0.1 mm Hg up to about 10 mm Hg. Because of the staggered arrangement of the connectors 18, the longitudinal ribs 12 are forced to bend or deform as the implant is circumferentially stretched, but the connectors 18 are relatively non-distendible and will not stretch by any appreciable amount. Preferably, connectors 18 near the base end of the harness are longer than the connectors near the apical end to provide a taper to the longitudinal ribs 12. (Alternatively, the connectors 18 could be made to have some elasticity and stretch somewhat as the heart expands and contract as the heart contracts, all the while the longitudinal ribs providing a continuous compressive force on the heart). Thus, the longitudinal ribs 12 must bend and provide compressive force on the heart. The FIGS. 6A and 6B at-rest cardiac harness 60 has a circumference that is governed by at least two structural variables: (1) the total number of longitudinal ribs 12 included in the structural design; and (2) the circumferential distance between adjacent longitudinal ribs 12. This latter variable can be subdivided into two parts: (1) the effective circumferential distance between adjacent longitudinal ribs; and (2) the circumferential length of the connectors 18 between adjacent ribs. Increasing either of these variables likewise increases the cardiac harness “at-rest” circumference. With respect to the longitudinal rib embodiments shown in FIGS. 5A-5H, the effective circumferential spring constant (stiffness) of cardiac harness 10 is governed by at least four structural variables: (1) the transverse cross-sectional configuration of the longitudinal ribs; (2) the flexural modulas of each longitudinal rib; (3) the longitudinal at-rest shape of each longitudinal rib; and (4) the longitudinal distance between the staggered connectors between adjacent ribs. In general, the cardiac harness' effective stiffness will increase as the longitudinal rib diameter and/or flexural modulas increases, and/or the distance between connectors 18 decreases. Further, the effect of the cross-sectional shape of the longitudinal ribs on the stiffness of the cardiac harness is shape specific.

An alternative embodiment of the cardiac harness shown in FIGS. 6A, 6B, 7A and 7B, are shown in FIGS. 7C-7K. In the cardiac harness 10 shown in FIGS. 7C-7K, the longitudinal ribs 12 extend from a base end 14 to the apical end 16 of the cardiac harness. Each of the embodiments has connectors 18 that have a different configuration and provide different spacing and flexing capabilities between the longitudinal ribs. As an example, as shown in FIGS. 7C and 7D, connector 18 has a pair of arms 70A that extend between longitudinal ribs 12. The pair of arms 70A are not connected to each and are curved and will provide a little flexibility and will straighten somewhat when tensioned. Similarly, referring to FIGS. 7E, 7F and 7G, the cardiac harness 10 has longitudinal ribs extending from the base end 14 to the apical end 16. The connectors 18 attach adjacent ribs and have a single curved arm 70B extending between the adjacent ribs. The arm 70B can flex slightly such that the curved portion will straighten slightly when tensioned. Referring to FIGS. 7H and 7I, the cardiac harness has longitudinal ribs 12 that extend from the base end 14 to the apical end 16. In this embodiment, the connectors 18 have a single arm 70D that extends between the adjacent longitudinal ribs 12. In this embodiment, arm 70D is substantially rigid, and will not flex or stretch as the longitudinal ribs bend as the heart contracts and expands. In another embodiment, shown in FIGS. 7J and 7K, the cardiac harness includes longitudinal ribs 12 that extend from base end 14 to apical end 16. In this embodiment, adjacent longitudinal ribs are connected by connectors 18 that have a curved arm 70E that can flex somewhat and straighten as adjacent longitudinal ribs expand and contract. Preferably, the connectors 18 of FIGS. 7C-7K are longer near the base end of the harness and get progressively shorter moving toward the apical end so that the longitudinal ribs 12 are tapered.

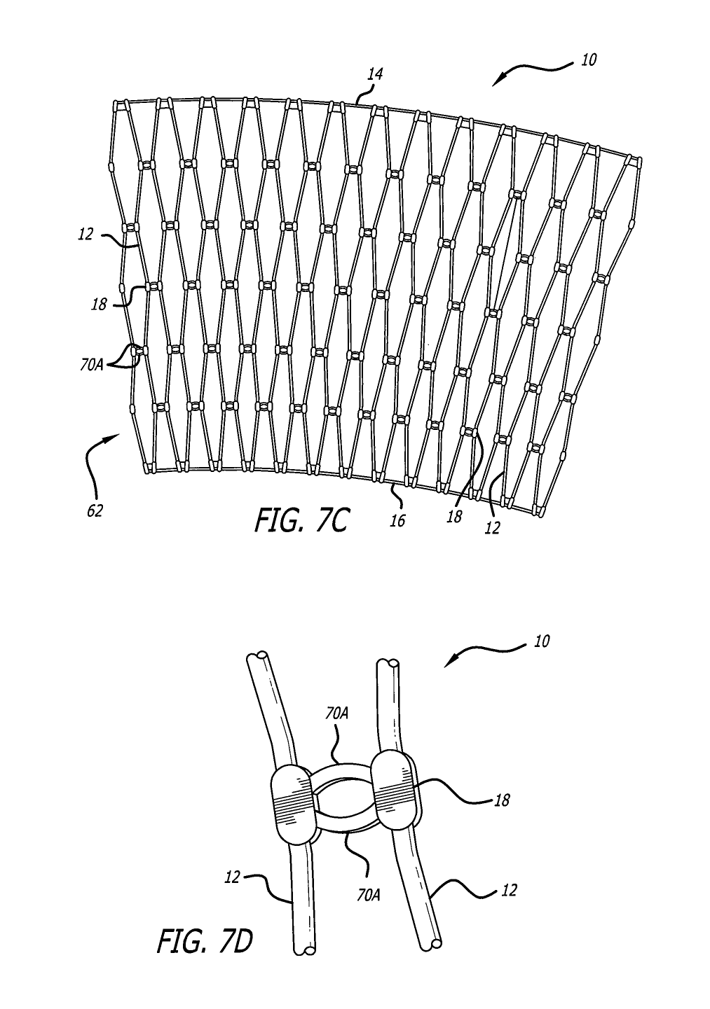

The longitudinal ribs 12 disclosed herein may have exposed ends that potentially could cause damage to surrounding tissue. Accordingly, and as shown in FIGS. 8A-8D, the ends are treated so as to minimize potential injury to surrounding tissue. More specifically, the end of longitudinal rib 12 as shown in FIG. 8A has an end cap 72 that can be formed of any type of atraumatic material, including silicone rubber, a soft and pliable polymer, or any other type of polymer. As shown in FIG. 8B, a ball-shaped cap 74 is formed on the end of the longitudinal rib 12 by, for example, laser melting the end of the metal alloy longitudinal rib 12 to form a ball-shaped cap 74. As shown in FIG. 8C, the end of longitudinal rib 12 can be bent into a loop end 76 in order to reduce trauma to the surrounding tissue. Likewise, as shown in FIG. 8D, the end of the longitudinal rib 12 can be bent into a closed loop end 78 to reduce the likelihood of damage to surrounding tissue. Moreover, the ends could be treated or embedded within an atraumatically-shaped rubber silicone coating, thereby providing additional protection to surrounding tissue.

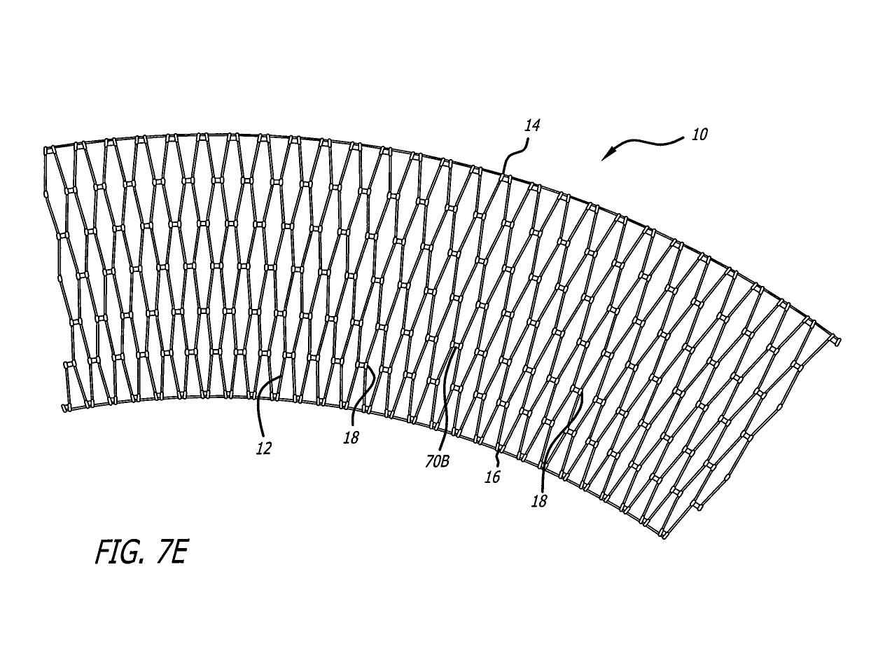

In one embodiment of the invention, as shown in FIG. 9, it is recognized that the cardiac harness 10 has a base end 14 and an apical end 16 with longitudinal ribs 12 connected by multiple connectors 18. In this embodiment, the ends of the longitudinal ribs may not generate significant circumferential compression forces along the ends due to the staggered configuration of the connectors 18 between the ribs. One method to increase the circumferential compressive force at the base end 14 and the apical end 16 of the cardiac harness 10 is to attach circumferentially oriented elastic bands 80 and 82 along the base and apex ends of the cardiac harness. Bands 80 and 82 are elastic and can be made from various materials including silicone rubber and should be matched to provide a compressive force that is consistent with the compressive force of the cardiac harness. For example, in the at-rest configuration, the elastic bands should be unstretched and provide no compressive force. When the cardiac harness is expanded, as shown in FIG. 9, the elastic bands should provide a compressive force consistent with the entire cardiac harness 10.

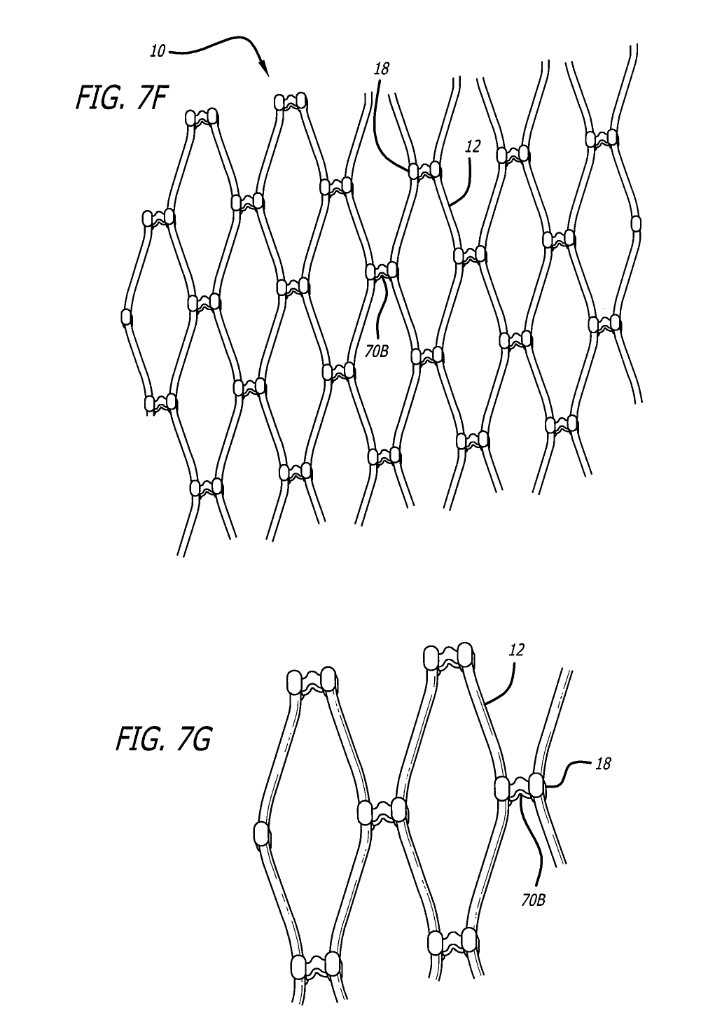

In alternative embodiments of the present invention, as shown in FIGS. 10A and 10B, the longitudinal ribs may have an at-rest shape that is different than the straight-wire shape shown in prior embodiments. For example, the at-rest shape of the longitudinal rib of FIG. 10A is a undulating longitudinal rib 90, while that in FIG. 10B is a sinusoidal or zig-zag-shaped at-rest longitudinal rib 92. Only one rib is shown in each FIGS. 10A-10B for ease of viewing. In these embodiments, the cardiac harness' final shape and performance can be altered by having a preformed shape with partial bends already imparted along the longitudinal rib, or as shown in FIG. 10A, to coordinate with the location of the connectors 18. These types of shapes may be advantageous for designs that, for example, desire to increase the at-rest circumference of the cardiac harness without significantly affecting the stiffness of the cardiac harness when it is expanded on the heart. The sinusoidal or zig-zag-shaped at-rest longitudinal rib 92 in FIG. 10B may actually decrease the stiffness of the cardiac harness (for a given wire diameter) because of the inherent flexibility of the bends as well as more evenly distributing bending stresses away from the connectors 18. Other specific longitudinal rib shapes may be designed to further optimize the spatial distribution of bending stresses in order to reduce the magnitude of localized peak bending stresses experienced by each rib, especially in the area of connectors 18.

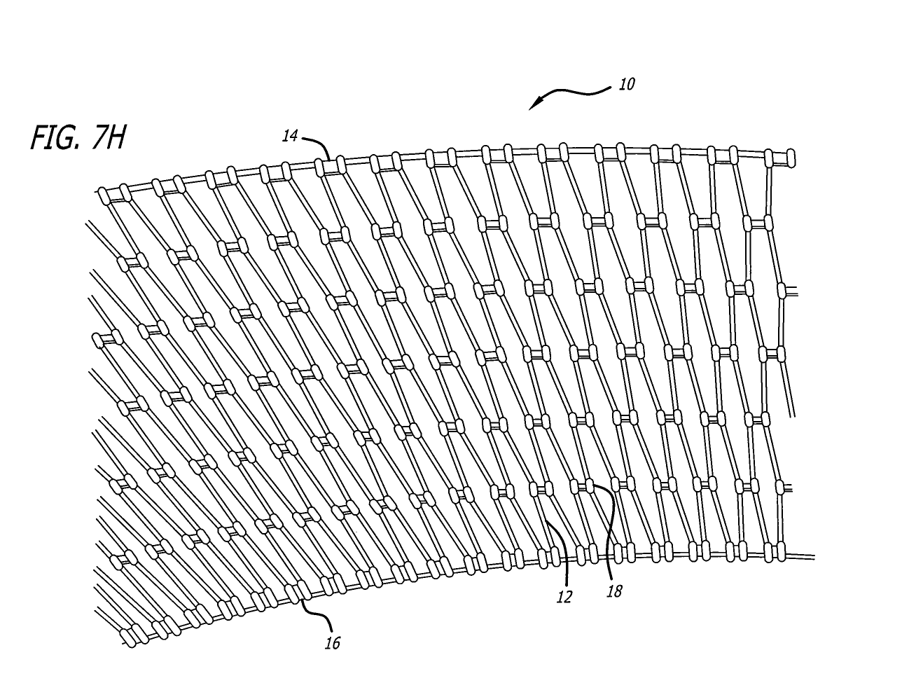

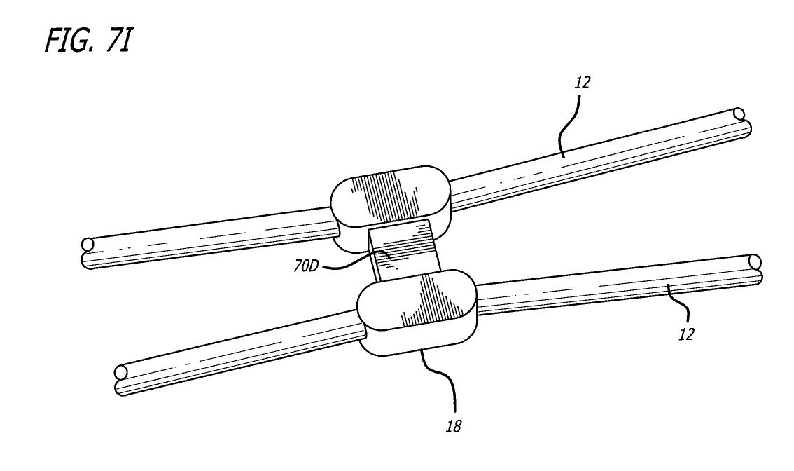

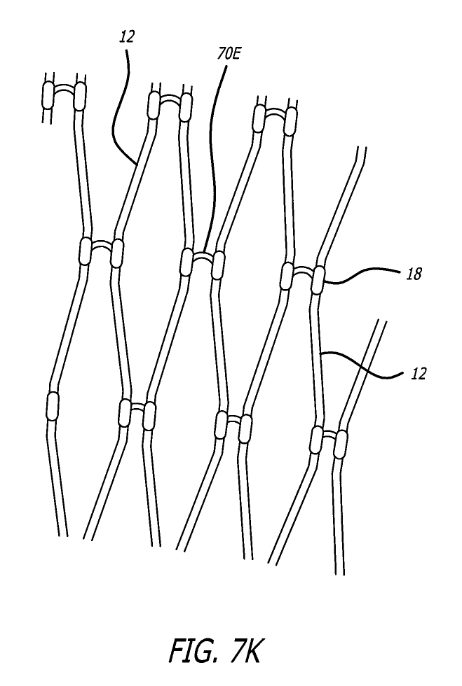

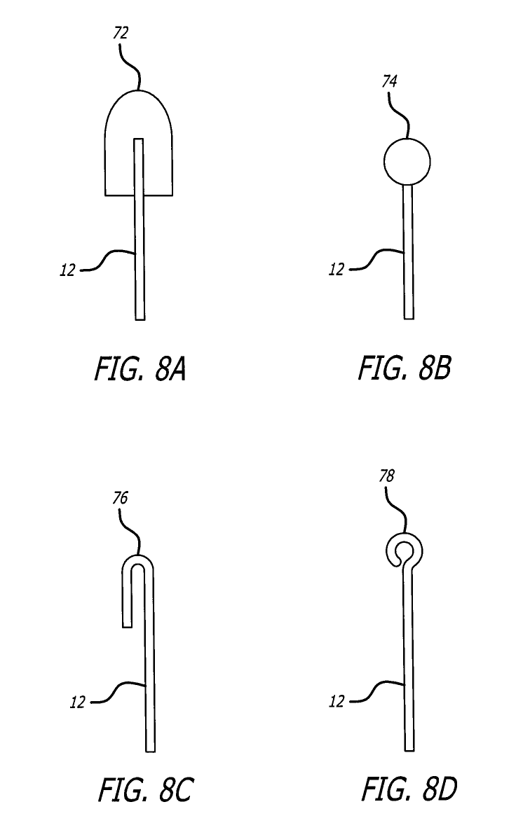

The present invention cardiac harness can be delivered to and mounted on the heart in a number of ways, including through a medium sternotomy or minimally invasive access. Prior art cardiac harness have been delivered minimally invasively through the ribs as disclosed in U.S. Pat. No. 6,602,184 and U.S. Pat. No. 7,189,203, both of which are incorporated herein by reference. Preferably, the cardiac harness of the present invention can be delivered minimally invasively through the ribs and through a small incision in the pericardium. In one embodiment, as shown in FIGS. 11-15, a tubular housing 100 has a housing lumen 102 and a central shaft 103. An at-rest cardiac harness 110 is compressed so that the harness fits between the housing lumen 102 and the central shaft 103. The longitudinal ribs 112 are compressed close together and the connectors 118 are flexible enough to bend and twist so that the harness can be compressed to a small diameter configuration.

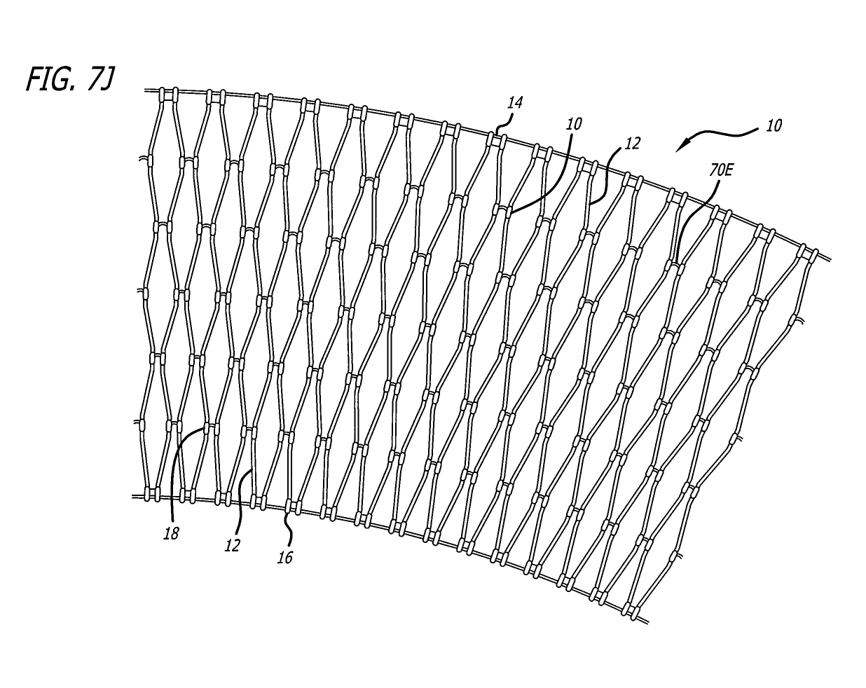

As shown in FIGS. 13-15, the compressed cardiac harness 110 is advanced out of the tubular housing 100 and onto the heart (not shown). In this embodiment, the cardiac harness was physically pushed out of the housing, however, other means for advancing the cardiac harness out of the housing are contemplated including a plunger, an actuator, or a device similar to that described in the aforementioned patents. Since the longitudinal ribs 112 of the cardiac harness 110 have substantial longitudinal column strength, it is likely that any type of pushing device or actuator is sufficient to push the harness onto the heart without the need for stabilizing the ribs as disclosed in the aforementioned prior art patents. In one embodiment (not shown), the tubular housing 100 can be configured to have a suction cup as disclosed in the aforementioned prior art patents in order to releasably attach the tubular housing to the heart during delivery of the cardiac harness.

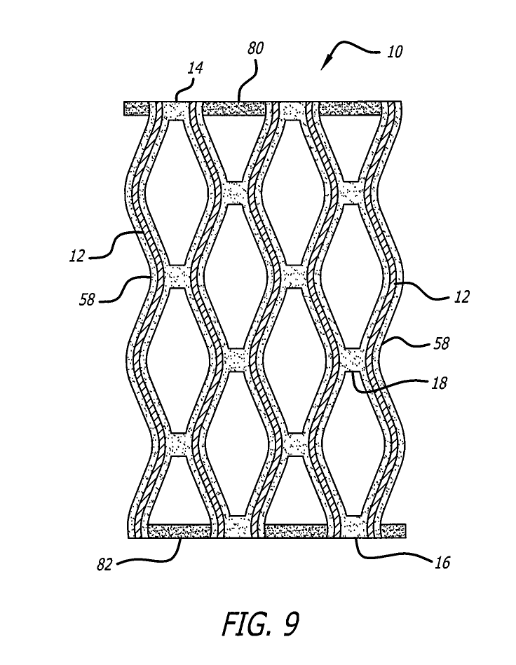

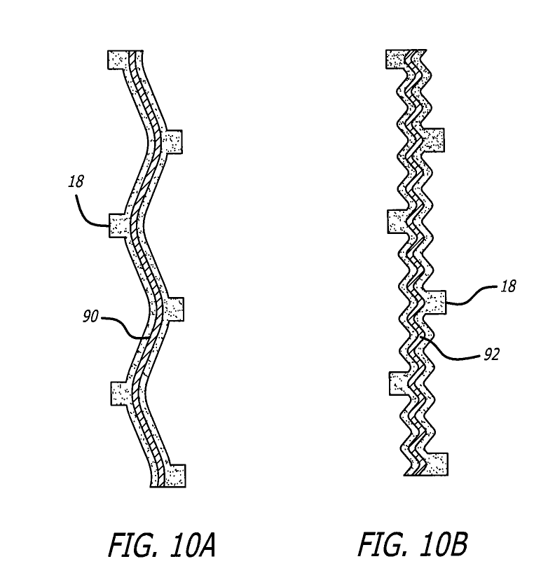

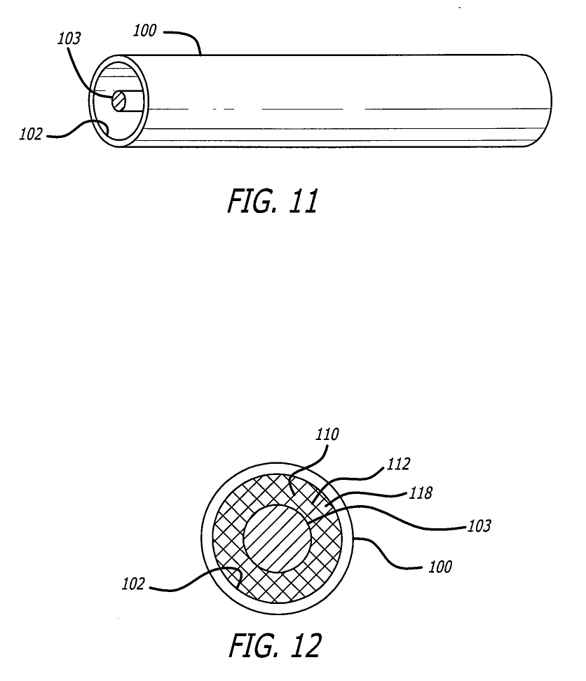

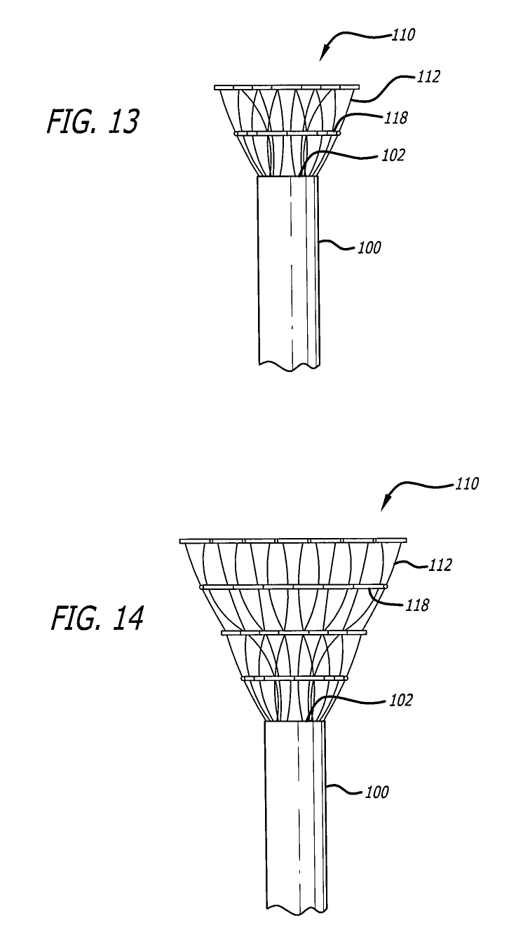

A prototype cardiac harness was built and is disclosed in FIGS. 16-19. As shown in FIGS. 16 and 17, an expanded cardiac harness 111 includes longitudinal ribs 112 constructed of segments of 0.1524 mm (0.006 inch) diameter nitinol wire (unpolished) inserted into connectors 118 comprising silicone tubing having 0.3048 mm (0.012 inch) inside diameter by 0.635 mm (0.025 inch) outside diameter. FIGS. 16 and 17 illustrate the cardiac harness 111 in a stretched or working condition, while FIGS. 18 and 19 illustrate an at-rest cardiac harness 110 in which the cardiac harness is in a collapsed condition.

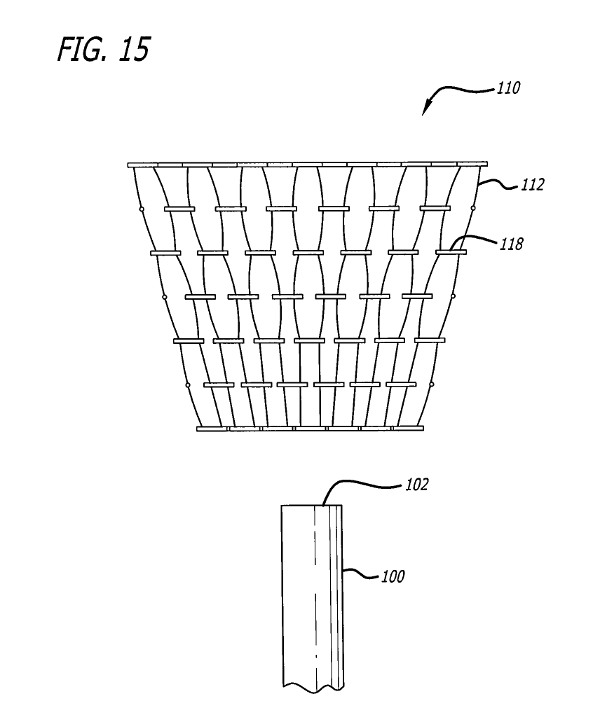

Any of the embodiments of the cardiac harness disclosed herein can be made by different means. In one embodiment, as shown in FIGS. 20A and 20B, the longitudinal ribs are placed in a mold and the connectors are molded directly onto the longitudinal ribs. Thus, as shown in FIG. 20A, the cardiac harness 120 includes longitudinal ribs 122 and curved connectors 124. The longitudinal ribs 122 are spaced apart in a parallel (or tapererd) orientation in the mold. The effective lengths of the curved connectors 124, which are overmolded onto the longitudinal ribs at specific points, are varied along the longitudinal dimension of the longitudinal ribs by introducing curvatures of specific amplitudes to the unstretched connectors 124. The curved connectors 124 at the base 125 of the cardiac harness 120 have a higher amplitude than the curved connectors near the apex 129 of the cardiac harness. So for example, base-curved connectors 128 have a higher amplitude of curvature than apex-curved connectors 130 positioned closer to the apex 129 of the cardiac harness. It is envisioned that, as the cardiac harness 120 is positioned over a heart, the curved connectors 124 will easily straighten so that their functional lengths are approximately equal to their associated unstretched path-lengths. Since the base-curved connectors 128 have a higher amplitude than the apex-curved connectors 130, the longitudinal ribs 122 will taper and be farther apart at the base 125 of the cardiac harness relative to the distance between the longitudinal ribs 122 at the apex 129 of the cardiac harness. This is illustrated in FIG. 20B in which the cardiac harness 120 is simulated as being mounted on a heart (not shown) wherein the longitudinal ribs are tapered and the curved connectors shown in FIG. 124 are now straightened connectors 126.

Importantly, the curved connectors 124 can be tailored to any desired form simply by designing the connectors with the appropriate path-lengths, which dictate the overall circumference of the cardiac harness 120 at any point along its length (i.e., longitudinal ribs 122). While FIG. 20A illustrates sinusoidal curves for the increasing curved connectors 124, any type of non-linear shape could be used to increase the functional path-length of each of the connectors. Further, a silicone rubber coating 132 can be molded at the same time onto the longitudinal ribs 122 in order to ensure that the cardiac harness 120 is electrically insulated from defibrillating or pacing functions as will be described herein. Elastic bands 134 can be incorporated at the base 125 and the apex 129 in order to connect the longitudinal ribs that do not have curve connectors 124 between the ribs at the ends. The number of ribs included in a mold can vary from at least two to as many ribs as is included in the cardiac harness, which in one embodiment includes 32 longitudinal ribs. In view of practical considerations, including mold sized, yield efficiency, inventory control, and the modularity between different sized cardiac harnesses, the actual number of longitudinal ribs included in a mold probably will be somewhere between 2 and 32. Further, in the illustrated embodiments, the longitudinal ribs 122 are straight ribs, however, this molding method is not restricted to straight ribs, and any rib shape should be amenable to the process, including undulating ribs, zig zag-shaped ribs, and the like. Also, while FIG. 20A shows the ribs in parallel alignment in the mold, the ribs could be aligned in a tapered manner in the mold, much like a hand-held fan.

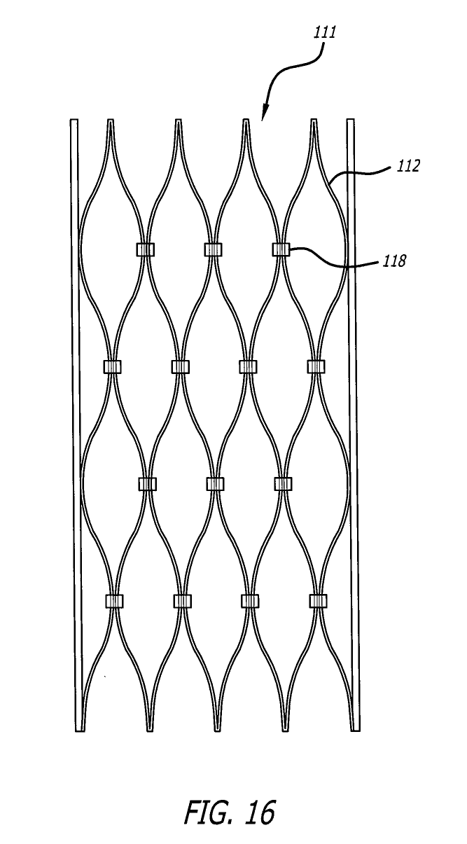

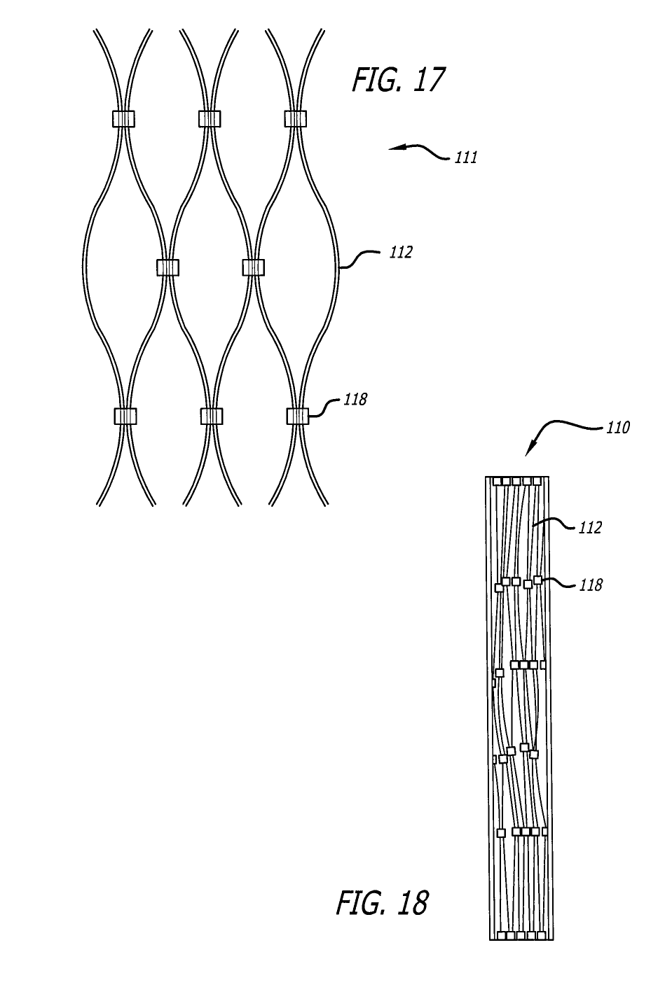

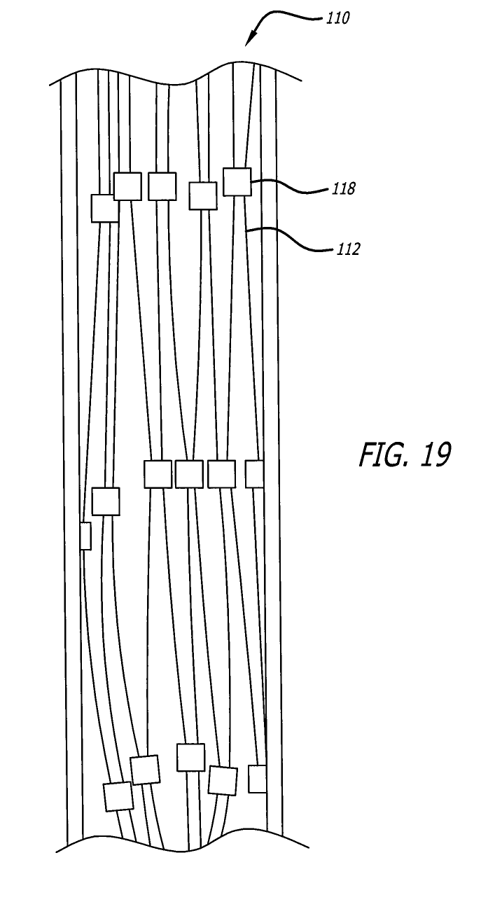

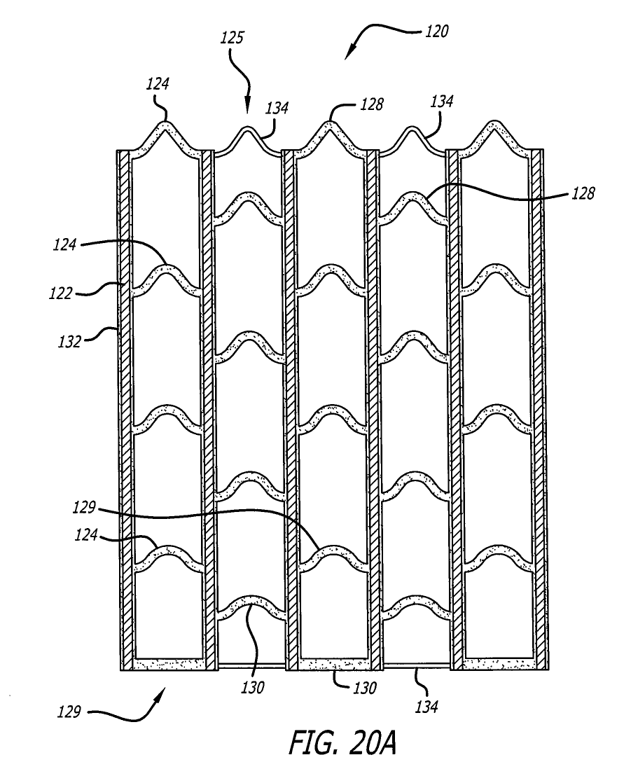

It is possible to equip the cardiac harness of the present invention with one or more epicardial pace/sense electrodes for monitoring the heart and/or pacing the heart in a known manner. It is important that the pace/sense electrodes be positioned on the harness so that the electrodes can provide an optimum benefit to the patient. Thus, in one embodiment of the present invention, as shown in FIGS. 21-24, a cardiac harness 140 has longitudinal ribs 142 that have a dielectric coating 143 on the ribs. The dielectric coating can include silicone rubber, or a similar material, so that the longitudinal ribs, that are formed from a metallic material such as nitinol, will be electrically insulated from the pace/sense electrodes. The longitudinal ribs are spaced apart by connectors 144 that also are formed of silicone rubber or a similar dieletric material. In this embodiment, pace/sense electrodes 146 are mounted on the cardiac harness 140 after the harness has been mounted on the heart. Each of the pace/sense electrodes 146 have a lead 148 that extends from the electrode to a power source. In order to mount the pace/sense electrodes 146 on the cardiac harness 140, guidelines 150 extend preferably from the base of the cardiac harness to the apex. A base guideline anchor 152 and an apex guideline anchor 154 are the terminal ends of the guidelines 150. Preferably, the guidelines 150 are parallel and fairly tight so that the pace/sense electrode 146 can be mounted onto the guidelines and slide along the guidelines in order to position the pace/sense electrodes 146 in relation to the epicardial surface of the heart. More specifically, guides 151 on the electrodes can be in form of pins, slots, grooves, or the like, which permit the pace/sense electrode to travel along the guidelines 150 and be positioned anywhere along the length of the cardiac harness. The guidelines 150 preferably remain free of attachments to the cardiac harness except at the base guideline anchor and apex guideline anchor 154.

Once the cardiac harness 140 has been mounted onto the heart, the surgeon or clinician will take an epicardial pace/sense electrode 146 (unipolar or bipolar) and insert the guidelines 150 into guides 151 on the electrode. Once engaged into the guidelines, the pace/sense electrode 146 can be manually and progressively pushed along the guidelines using the lead 148 which has some column strength to push and pull along the guidelines. The clinician can stop at any position along the longitudinal path of the guidelines 150 to evaluate the pace/sense signal quality in order to determine an optimal or desired electrode location. If a desirable location is not found along a particular guideline path, the pace/sense electrode can be withdrawn and disengaged from the guidelines, and then reintroduced along a different pair of guidelines on the cardiac harness 140 for further testing. Once a desired location is determined, the pace/sense electrode 146 can be optionally further secured at the relative location by one of a number of potential, passive or active means.

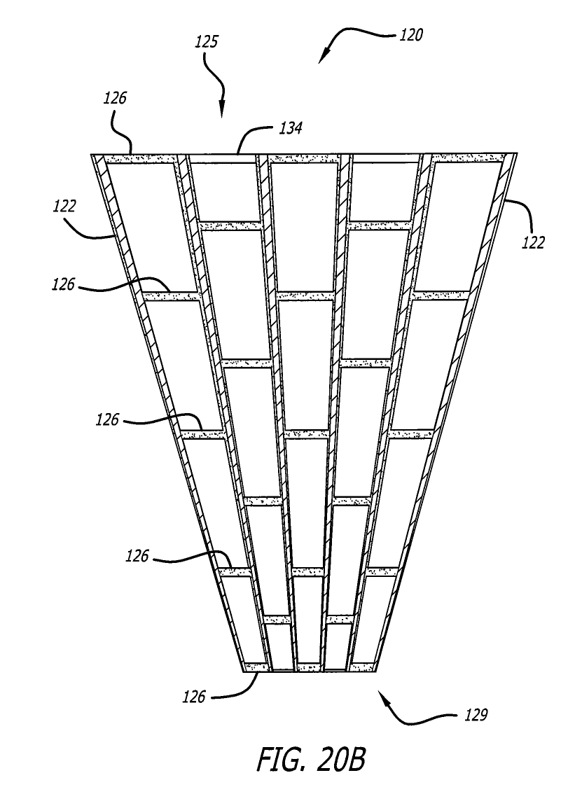

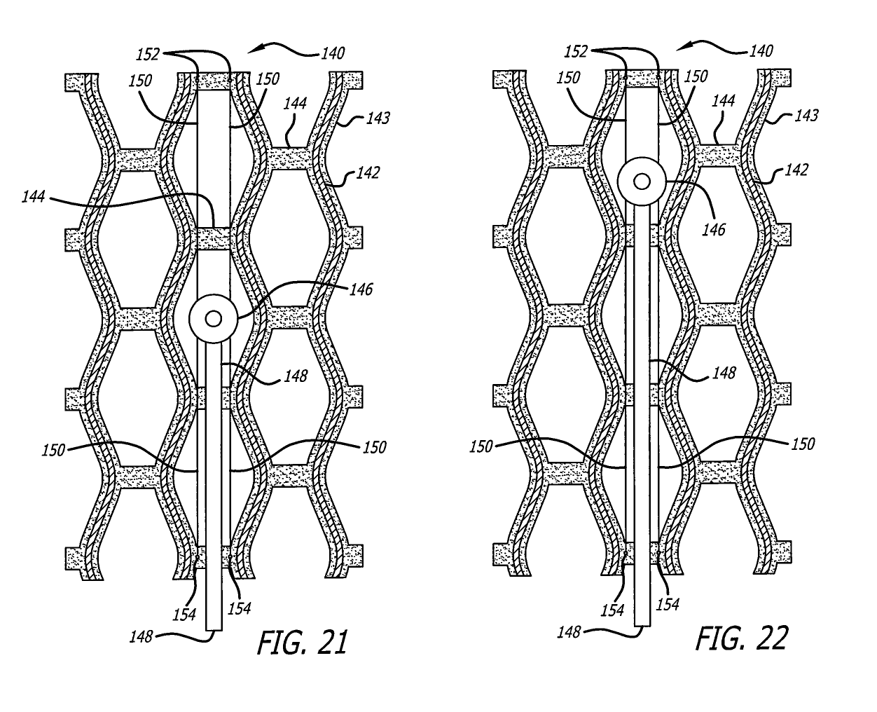

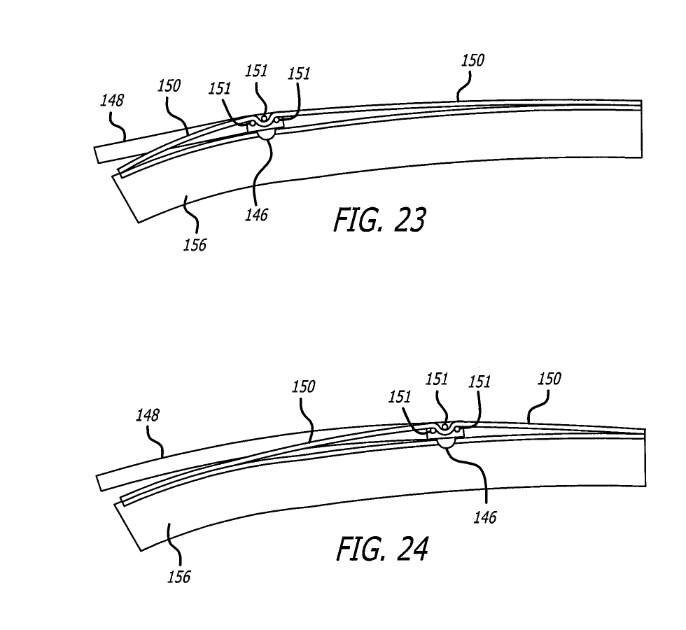

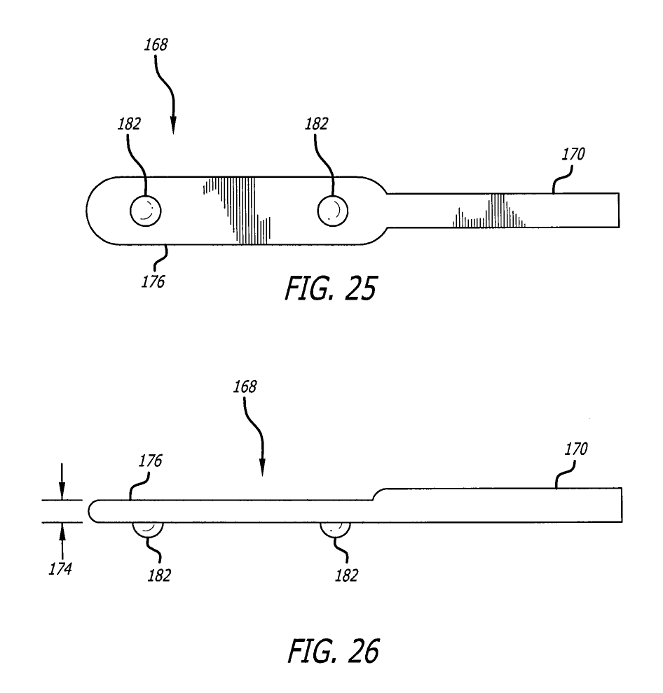

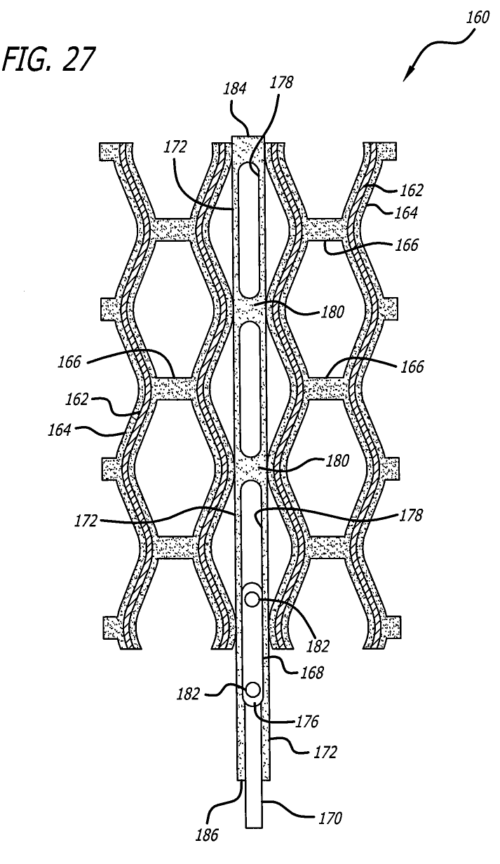

Another embodiment where epicardial pace/sense electrodes can be installed onto a cardiac harness after the harness has been deployed onto the heart is shown in FIGS. 25-29. In this embodiment, a cardiac harness 160 includes longitudinal ribs 162 having a dielectric coating 164 so that the longitudinal ribs, which are formed of a metal alloy (nitinol) are electrically insulated. The longitudinal ribs 162 are spaced apart by connectors 166 as previously described. Preferably, the connectors also are formed of a dielectric material such as silicone rubber. One or more bipolar pace/sense electrodes 168 are removably attached to the cardiac harness 160 after the harness is mounted on the heart. The pace/sense electrodes 168 have leads 170 that extend from the pace/sense electrode to a power source (not shown). In this embodiment, guide sheaths 172 are molded to align with the longitudinal ribs 162 and extend in a parallel fashion between adjacent longitudinal ribs. The pace/sense electrodes 168 have a flattened distal section 174 in the shape of a paddle 176. The guide sheaths 172 have openings 178 that correspond to the shape of paddle 176. The guide sheaths 172 extend the entire length of the cardiac harness and can be attached at multiple locations along the length of the longitudinal ribs 162 by molding (or gluing) the guide sheaths 172 directly to the longitudinal ribs 162. Alternatively, the guide sheaths can be attached separately to a completed harness by overmolding or gluing the guide sheaths onto existing connectors 166 of the harness. The openings 178 in the guide sheaths 172 are separated by spacers 180 which can be molded directly to the longitudinal ribs 162 and act as connectors 166 to space the longitudinal ribs, and help attach the guide sheaths 172 to the cardiac harness 160. After the cardiac harness 160 has been mounted on the heart, the surgeon or clinician can advance a pace/sense electrode 168 along any of the openings 178 in order to determine the optimum position of the pace/sense electrode 168 on the heart. As an example, the pace/sense electrode 168 can be positioned in an opening 178 near the apex of the heart, tested, and moved to a different opening 178 if the tests show that a more optimum position on the heart is available. The pace/sense electrode 168 should make direct contact with the myocardium, thus the pace/sense electrodes may have a single pace/sense button electrode (unipolar) or a closely spaced (approximately 1 cm apart) pair of pace/sense button electrodes (bipolar) positioned on the flattened distal section 174. The single or pair of pace/sense button electrodes 182 extend from the flattened distal section 174 and will come in direct contact with the myocardium when the pace/sense electrodes are positioned in openings 178. It is preferred that all portions of the guide sheaths 172 be porous to ionic fluids such that, especially when not used, it presents minimal electrical resistance to the passage of defibrillation currents (especially from external defibrillation) in the event the patient has a cardiac episode requiring defibrillation. The pace/sense electrodes 168 can either be steroid eluting or non-steroid eluting electrodes, depending upon the particular need. If the electrode is steroid-eluting, it will reduce the likelihood of the buildup of fibrosis around the electrode.

In one embodiment, the cardiac harness 160 is mounted on the heart and a pace/sense electrode 168 is inserted as desired into the guide sheath 172 at one of the openings 178. Care should be taken to make sure that prior to insertion of the pace/sense electrode that it is rotated so that the pace/sense button electrodes 182 are facing inwards toward the myocardium. The proximal end of the lead 170 (not shown) may be optionally connected to a pace stimulation analyzer (PSA), an implantable pulse generator (with our without multi-site pacing capabilities, e.g., ICD-CRT, pacemaker), or similar device to enable dynamic testing of the pace/sense electrode 168 within a desired region or at various sites along the guide sheath 172. The pace/sense electrode 168 is advanced along the guide sheath 172 and inserted at various openings 178, with testing with a PSA (or equivalent) to determine the robustness of the contact with the myocardium (e.g., sense amplitude, pace capture thresholds) and to determine the suitability of the relative positioning within the sheath of the electrodes on the heart in order to provide optimized CRT pacing. If a position is determined to be not adequate, the pace/sense electrode 168 can be advanced or retracted along the guide sheath 172 into various of the openings 178 until an optimum opening is determined. Once an acceptable position is identified along the guide sheath 172, it may be desirable to secure the pace/sense electrode 168 in opening 178. Several means are available to attach the pace/sense electrode 168 to opening 178 including cinching one or more sutures around the guide sheath 172 at the opening 178 in order to capture the pace/sense electrode 168. Alternatively, an inflatable balloon (not shown) may be incorporated on lead 170 just behind the pace/sense electrode 168 where the balloon would remain deflated until the pace/sense electrode 168 was optimally placed in opening 178. The balloon could then be inflated whereby it would push against the wall of the guide sheath 172 on one side, and the pericardium on the other side in order to force the lead 170 and pace/sense electrode 168 into the opening 178. Further, the inflated balloon would ensure that the pace/sense button electrodes 182 are pushed firmly against the pericardium, thereby improving electrode contact and functional performance. If an acceptable position is not identified within a particular guide sheath 172, the pace/sense electrode 168 can be completely removed from one guide sheath and redeployed within an alternative guide sheath in order to continue to search for a desirable pace/sense location. If unipolar pace/sense electrodes are used, two separate electrodes can be independently positioned along separate (preferably neighboring) guide sheaths 172 to create (with an appropriate Y connector) a functional bipolar system. Because the pace/sense electrode 168 is physically independent of the cardiac harness 160, it can be manufactured separately from and independent of the harness, although it may be advantageous to coordinate its design with that of the cardiac harness and guide sheaths 172.

The present invention method of use enables “atrial epicardial” pace/sense electrode(s) to be installed onto the cardiac harness after the harness has been deployed onto the heart. In particular, it is envisioned that each guide-sheath 172 (see FIGS. 25-29) described for use with a ventricular pace/sense electrode be used, except that the base end 184 of the guide-sheath 172 would remain open (unsealed) with at least the same diameter as the remainder of the sheath.

One method by which this system would be used procedurally is envisioned to be as follows. The cardiac harness 160 with guide-sheaths 172 is deployed onto the heart via appropriate means. Once the cardiac harness is acceptably deployed, an atrial pace/sense electrode 168 (described further below) is inserted into the open apical end 186 of guide-sheath 172. The preferred guide-sheath is chosen to be that one which is estimated/expected to provide the best alignment with the atrium (e.g., epicardial surface of the RA or LA appendage) from which it is desired to pace and/or sense.

The lead 170 of the pace/sense electrode 168 may be optionally (but perhaps preferably) connected to a pace stimulation analyzer (PSA), implantable pulse generator (with or without multi-site pacing capabilities; e.g., ICD-CRT, pacemaker), or similar to enable testing of the pace/sense electrode (once it is placed).

The atrial pace/sense electrode 168 is advanced along the guide-sheath 172 until it exits the base end 184 of the sheath. The distal end of the pace/sense electrode is then advanced further (perhaps with visual/electrogram guidance and with stylet assistance) and directed towards the atrial epicardium (e.g., RA or LA appendage). Once contact with the atrial epicardium is established, the electrode's active fixation mechanism (e.g., helical tip) is activated to positively engage the epicardium. A more passive means can include simple friction or use of a balloon to press the electrodes onto the epicardium.

With the pace/sense electrode actively fixed into the atrial epicardium, the electrode may be tested with a PSA (or equivalent) to (a) confirm fixation into atrial (versus ventricular or non-myocardial) tissue, and (b) determine the robustness of the contact with the epicardium (e.g., sense amplitudes, pace capture thresholds, etc.). If the position is determined to be inadequate (e.g., poor thresholds, sensed R-waves too large, etc.), the pace/sense electrode 168 can be disengaged from the atrium, repositioned as desired, and re-engaged, at which point electrode testing can be repeated.

Once an acceptable position is identified within the guide-sheath 172, it may be desirable to (optionally) secure the electrode in place within the sheath. For example, the user could cinch one or more sutures around the base end 184 of the sheath such that the electrode 168 is captured within the sheath. To protect the lead from potential damage (direct or indirect) from the suture, it may be further desirable to use a suture sleeve over the sheath, or directly incorporate a suture-sleeve-like strain relief onto the base end 184 of the sheath itself.

If an acceptable position is not identified/attainable within this sheath 172, the electrode can be completely removed from the sheath and then redeployed (as described above) within an alternate sheath (if available) on the cardiac harness to continue to search for a desirable atrial pace/sense location.

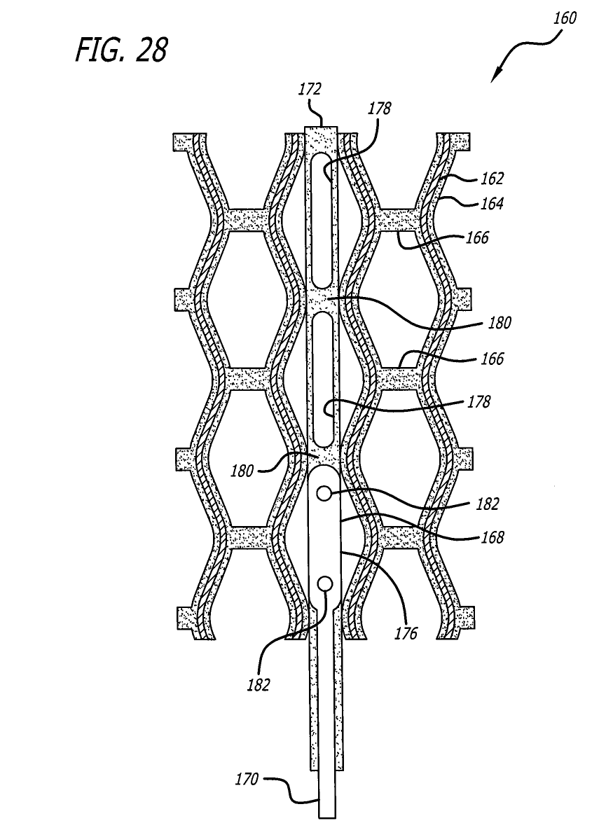

In further keeping with the invention as illustrated in FIGS. 30-34, the cardiac harness of the present invention can be used to position defibrillating electrodes at various locations on the heart. In some prior art devices, the defibrillating electrodes are incorporated into the cardiac harness prior to mounting the harness onto the heart. This provides a secure means for attaching the electrodes to the cardiac harness, but also increases the profile of the harness when compressing the harness into a housing in order to deliver the harness minimally invasively between the ribs and through an opening in the pericardium on the heart. The present invention provides for adding the defibrillating electrodes onto the cardiac harness after the harness has been mounted on the heart. As previously described, the cardiac harness 190 includes longitudinal ribs 192 that are coated 194 with a dielectric material such as silicone rubber. The longitudinal ribs are connected by connectors 196, which also are formed of a dielectric material such as silicone rubber or the like. Thus, the longitudinal ribs 192 and the connectors 196 are electrically insulated, since the longitudinal ribs 192 preferably are made from a metallic alloy such as nitinol.

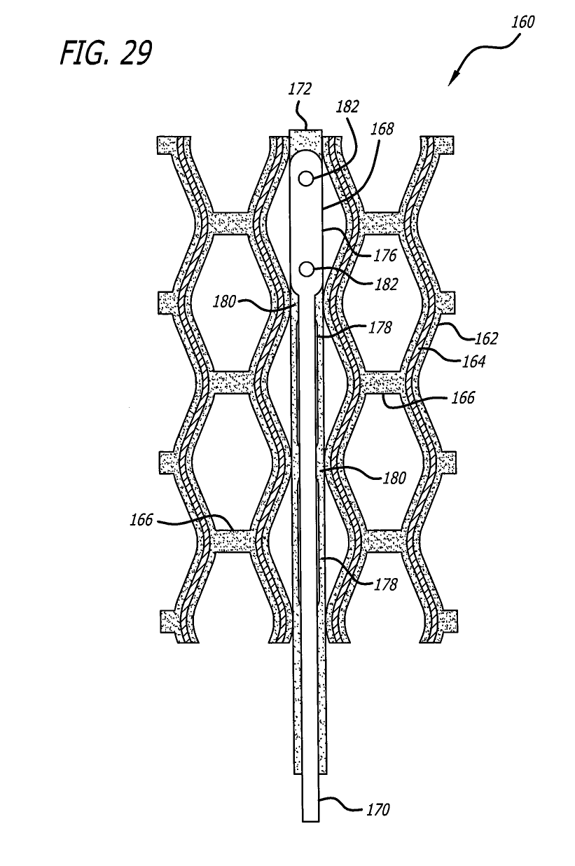

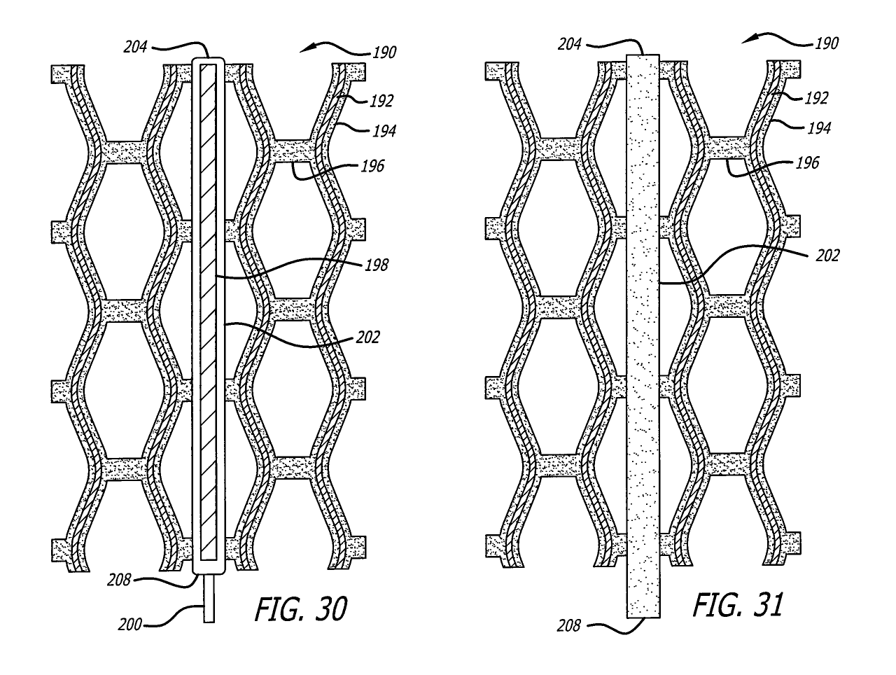

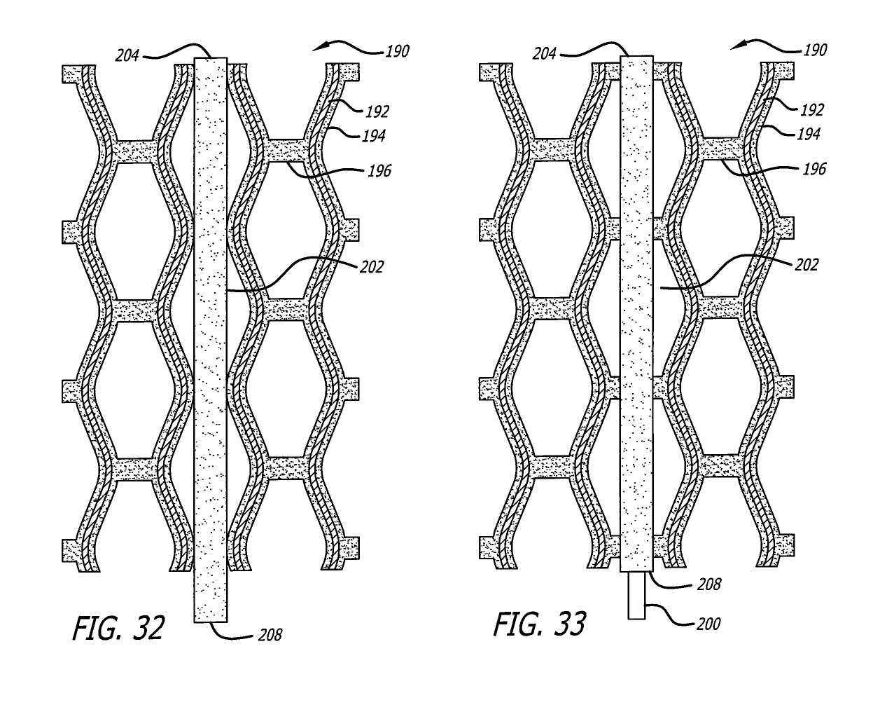

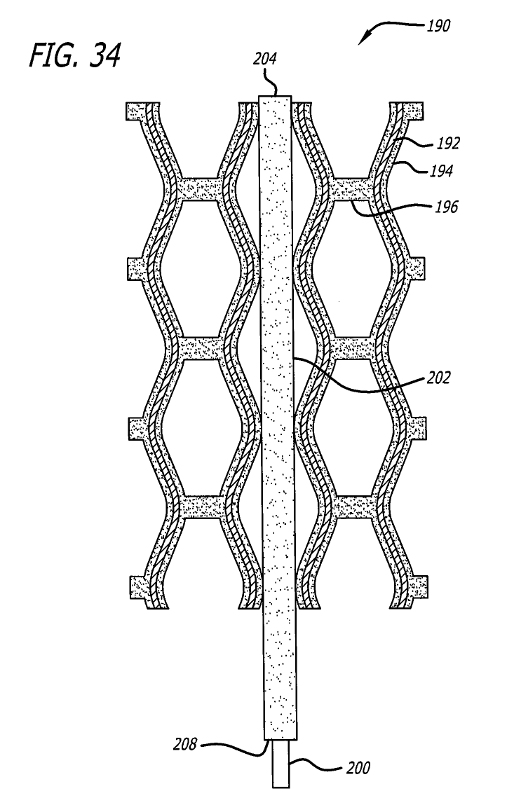

In keeping with the invention, a guide sheath 202 is incorporated into the cardiac harness 190 and is configured to receive a defibrillation electrode 198 with a lead 200 attached to the electrode. The guide sheath 202 extends from base end 204 of the sheath to apical end 208 of the sheath which corresponds to the full length of the harness which extends from the base end of the harness to the apical end of the harness. Multiple guide sheaths 202 can be positioned along the circumference of the cardiac harness in order to ensure the optimum positioning of defibrillation electrodes once inserted. In one embodiment as shown in FIGS. 31 and 33, the guide sheath 202 is molded into and in line with the longitudinal ribs with an orientation that is parallel to the ribs and interconnected to adjacent ribs via symmetric non-staggered connectors 196. A defibrillation electrode and lead 200 are inserted in the guide sheath 202 (FIG. 33). Alternatively, the guide sheath 202 can be securely attached to the pericardial side of a longitudinal group of connectors 196 on an otherwise previously mounted cardiac harness 190 (see FIG. 32). In FIG. 34, a defibrillation electrode and lead 200 have been positioned in the guide sheath 202 of FIG. 32. Each embodiment of guide sheath 202 should have basic characteristics including being thin walled and very flexible and collapsible, such that it occupies very little volume when collapsed. Further, it may or may not be extensible (in either length or diameter) depending upon design intent. Also, guide sheath 202 should be open on its apical end 208 and closed on its base end 204. The diameter of guide sheath 202 should be configured to accept the diameter of the defibrillation electrode 198 so that the electrode can be advanced or pushed into the guide sheath easily and without much friction. The apical end 208 of guide sheath 202 may optionally be flared to larger size to more easily receive the defibrillation electrode 198. At least a portion of the guide sheath 202 is formed of a porous material, which is porous to ionic fluids such that it presents minimal electrical resistance to defibrillation currents. Examples of such materials to form the guide sheath 202 include woven meshes of an appropriate biocompatible material, ePTFE tubing with appropriate pore sizing.

Once the cardiac harness 190 has been mounted onto the heart, the defibrillation electrodes 198 are fully advanced into the guide sheath 202 by hand, since the defibrillation electrode 198 will have some column strength so that the electrode can be advanced into the guide sheath 202. Optionally, a releasable mandrel or guide wire can be attached to the defibrillation electrode to help advance it into the guide sheath 202. After the defibrillation electrode is fully advanced into the guide sheath, the mandrel or guide wire can be released and withdrawn from the guide sheath 202. If the apical end 208 of the guide sheath 202 is flared, it will assist the clinician or surgeon in initially locating the opening to the guide sheath and advancing the defibrillation electrode 198 into the sheath. Optionally, the defibrillation electrode 198 may be subsequently secured in place within the guide sheath 202 by, for example, cinching one or more sutures around the proximal section (apical end 208) of the sheath such that the electrode is captured within the sheath. In order to protect the electrode from potential damage from the suture, it may be further desirable to use a suture sleeve over the sheath, or directly incorporate a suture sleeve-like strain relief onto the proximal end of the sheath itself.

Because the defibrillation electrode is physically independent of the cardiac harness, it can be manufactured separately from and independently of the harness, although it may be advantageous to coordinate the design of the electrode with that of the guide sheath 202. For example, the defibrillation electrode 198 could be designed with a diameter change (or notch) just proximal to the defibrillation coil that would interface with a diameter change (narrowing) within the guide sheath to help automatically secure the electrode within the sheath once it is properly and fully advanced into the sheath. As another example, at least some portions of the electrode, particularly the portion that includes the defibrillation electrode, could have a flattened or non-circular cross-section (i.e., an oval or elliptical or more flattened cross-section).

In order to maintain adequate defibrillation impedances, the guide sheath 202 could be manufactured to ensure that the pericardial side of each guide sheath 202 is made effectively non-porous, thereby limiting or restricting current to flow only through the epicardial aspect. Alternatively, the porosity can be varied along the longitudinal length of the guide sheath. This spatial variation can be tailored to enable a “shaping” of the current density distribution along each electrode during defibrillation (e.g., to minimize electrode “edge effects” or to direct relatively more current towards the base than the apex, or vice versa).

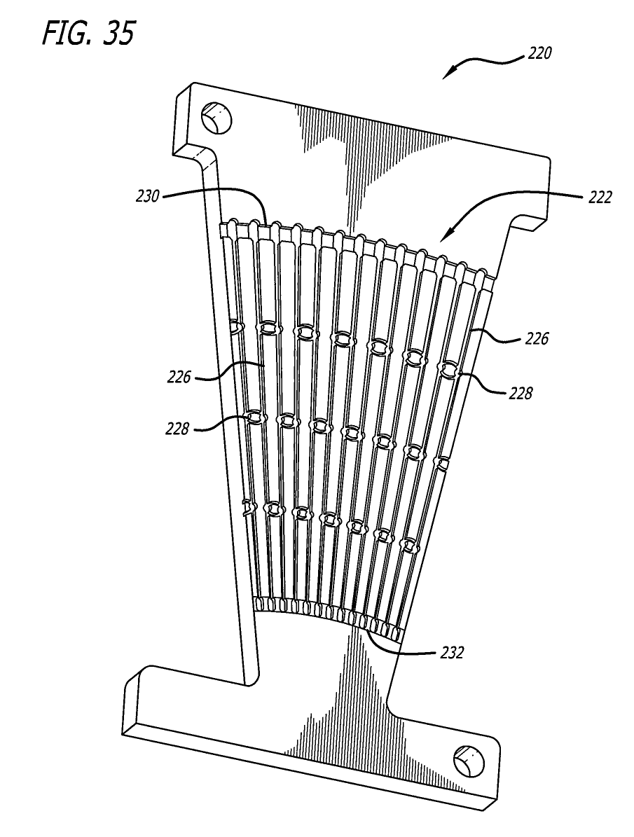

In further keeping with the invention and as shown in FIG. 35, a mold 220 is used to form at least a portion of the cardiac harness 222. In this embodiment, a portion of the cardiac harness 222 is formed in the mold, and after several sections are made, the sections can be connected in order to form a completed cardiac harness. Longitudinal ribs made of nitinol wire (not shown) are placed in longitudinal rib cavities 226 in the mold 220. Thereafter, a mirror image of mold 220 are fastened together and a polymer injected into the mold in order to flow the polymer into connector cavity 228, base end cavity 230, and apical end cavity 232. Further, the polymer will flow into the longitudinal rib cavity and surround the longitudinal ribs formed from nitinol. In one embodiment, the polymer injected into the longitudinal rib cavity 226 may be different from the polymer injected into the connector cavity 228, and different yet from the polymer injected into the base end cavity 230 and the apical end cavity 232. As can be seen with reference to FIG. 34, the spacing of the connector cavities 228 at the base end of mold 220 are farther apart than the apical end cavities 232, so that the longitudinal ribs are tapered relative to each other. Tapering the ribs is preferred since the base end of the heart is larger than the apex end of the heart, requiring a tapering from a larger spacing between longitudinal ribs near the base end, to a narrower spacing between the longitudinal ribs at the apex end of the cardiac harness.

It may be desired to reduce the likelihood of the development of fibrotic tissue around the cardiac harness which may increase stiffness and thereby change the desired compressive force of the harness on the heart. Certain drugs such as steroids, have been found to inhibit cell growth leading to scar tissue or fibrotic tissue growth. Examples of therapeutic drugs or pharmacologic compounds that may be loaded onto the cardiac harness or into a polymeric coating (silicone rubber) on the cardiac harness or infused into the area surrounding the harness include steroids, taxol, aspirin, prostaglandins, and the like. Various therapeutic agents such as antithrombogenic or antiproliferative drugs are used to further control scar tissue formation. Examples of therapeutic agents or drugs that are suitable for use in accordance with the present invention include 17-beta estradiol, sirolimus, everolimus, actinomycin D (ActD), taxol, paclitaxel, or derivatives and analogs thereof. Examples of agents include other antiproliferative substances as well as antineoplastic, antiinflammatory, antiplatelet, anticoagulant, antifibrin, antithrombin, antimitotic, antibiotic, antimicrobial and antioxidant substances. Examples of antineoplastics include taxol (paclitaxel and docetaxel). Further examples of therapeutic drugs or agents include antiplatelets, anticoagulants, antifibrins, antiinflammatories, antithrombins, and antiproliferatives. Examples of antiplatelets, anticoagulants, antifibrins, and antithrombins include, but are not limited to, sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor (available from Biogen located in Cambridge, Mass.), and 7E-3B® (an antiplatelet drug from Centocor located in Malvern, Pa.). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen located in the United Kingdom), angiotensin converting enzyme inhibitors such as Captopril® (available from Squibb located in New York, N.Y.), Cilazapril® (available from Hoffman-LaRoche located in Basel, Switzerland), or Lisinopril® (available from Merck located in Whitehouse Station, N.J.); calcium channel blockers (such as Nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug from Merck), methotrexate, monoclonal antibodies (such as PDGF receptors), nitroprusside, phosphodiesterase inhibitors, prostaglandin inhibitor (available from GlaxoSmithKline located in United Kingdom), Seramin (a PDGF antagonist), serotonin blockers, steroids, thioprotease inhibitors, triazolopyrimidine (a PDGF antagonist), and nitric oxide. Other therapeutic drugs or agents which may be appropriate include alpha-interferon, genetically engineered epithelial cells, and dexamethasone. It may also be desirable to incorporate osteogenic or angiogenic factors with the cardiac harness to promote healing.

Although this invention has been disclosed in the context of several preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed:

1. A cardiac harness, comprising:
a plurality of longitudinal ribs spaced apart and having a base end and an apex end;
a plurality of connectors, the connectors being configured to attach adjacent longitudinal ribs to each other, the connectors extending only between adjacent ribs; and
the longitudinal ribs having a high degree of longitudinal flexibility so that when the cardiac harness is mounted on the heart, the longitudinal ribs flex along a longitudinal axis of the ribs as the heart expands and contracts circumferentially.

2. The cardiac harness of claim 1, wherein the connectors are substantially nondistensible.

3. The cardiac harness of claim 1, wherein the connectors are flexible.

4. The cardiac harness of claim 1, wherein the longitudinal ribs are formed from a superelastic material and the connectors are formed from a polymer material.

5. The cardiac harness of claim 4, wherein the longitudinal ribs are formed from at least one nitinol wire having a diameter in the range of about 0.127 mm to about 0.762 mm (0.005 inch to about 0.030 inch).

6. The cardiac harness of claim 4, wherein the polymer forming the connectors is taken from the group of polymers consisting of silicone rubber, nylons, polyethylene, polypropylene, fluorospolymers and polyurethane.

7. The cardiac harness of claim 1, wherein the connectors can have different sizes and shapes including a bar, a tube, a ring, a sinusoidal segment, a straight segment, an undulating segment, a straight and an undulating segment, a zig zag segment, a bifurcated segment, and a curved segment.

8. The cardiac harness of claim 1, wherein the spacing between adjacent ribs at a base portion of the cardiac harness is greater than the spacing between adjacent ribs at an apex portion of the cardiac harness so that the spacing between the ribs tapers down from the base portion toward the apex portion.

9. The cardiac harness of claim 1, wherein the longitudinal ribs can have different shapes including straight, curved, sinusoidal, and undulating, and different transverse cross-sections including cylindrical, oval, rectangular, square, and I beam.

10. The cardiac harness of claim 9, wherein the cardiac harness has an at-rest configuration and an expanded configuration, the longitudinal ribs being generally undeformed in the at-rest configuration and generally deformed in the expanded configuration.

11. The cardiac harness of claim 1, wherein the cardiac harness has an at-rest configuration and an expanded configuration, the longitudinal ribs being generally undeformed in the at-rest configuration and generally deformed in the expanded configuration.

12. The cardiac harness of claim 1, wherein the longitudinal ribs are coated with dielectric material taken from the group of materials consisting of silicone rubber, polyurethane, parylene, polyester, polyimide, fluoropolymers, and similar materials.

13. A method of treating the heart, comprising:
providing a cardiac harness having a plurality of longitudinal ribs spaced apart and a plurality of connectors attaching adjacent ribs together;
the cardiac harness having an at-rest configuration and a first circumference C₁, and an expanded configuration when mounted on the heart at end diastolic filling, the expanded configuration defining a second circumference C₂;
C₁ being substantially less than C₂; and
the longitudinal ribs elastically deforming with a spring-like force as the cardiac harness expands from the C₁ configuration to the C₂ configuration.

14. The method of claim 13, wherein the cardiac harness applies a compressive force on the heart in the C₂ configuration at end diastolic filling in the range of about 0.5 mm Hg to about 10 mm Hg.

15. The method of claim 13, wherein the longitudinal ribs are formed from a superelastic material and provide a substantially uniform compressive force on the heart throughout the cardiac cycle.

16. The method of claim 13, wherein the connectors have any of lengths L₁, L₂, L₃, up to Lₙ, and wherein as the cardiac harness expands from the C₁, configuration to the C₂ configuration, the length L₁, L₂, L₃, up to Lₙ remain substantially the same.

17. A method of mounting a cardiac harness on a heart, comprising:
providing a cardiac harness having a plurality of flexible longitudinal ribs spaced apart and a plurality of connectors attaching adjacent ribs together;
compressing the cardiac harness into a tubular housing;
positioning the tubular housing through a minimally invasive access site in the patient so that the tubular housing is proximate the apex of the heart;
advancing the cardiac harness out of the tubular housing and over the apex of the heart;
further advancing the cardiac harness onto the heart so that the cardiac harness is mounted on the heart and covers a substantial portion of the heart; and
wherein the flexible longitudinal ribs provide column strength to the cardiac harness as the harness is advanced onto the heart.

18. The method of claim 17, wherein the connectors maintain a predetermined spacing between adjacent longitudinal ribs.

19. The method of claim 17, wherein a suction cup assembly associated with the tubular housing is releasably attached to the apex of the heart via a vacuum so that the suction cup assembly can be pulled slightly proximally thereby pulling on the apex and placing the heart in tension and elongating the heart slightly to more easily allow the cardiac harness to advance onto the heart.

20. A method of making a cardiac harness, comprising:
imparting a predetermined at-rest shape to a plurality of superelastic longitudinal ribs;
electropolishing the longitudinal ribs;
cutting the longitudinal ribs to a predetermined length;
position the plurality of longitudinal ribs in an at-rest pattern between two sheets of silicone rubber;
vulcanize the two sheets of silicone rubber together thereby entrapping the ribs;
removing excess silicone rubber so that a plurality of connectors are formed between adjacent longitudinal ribs.

21. The method of claim 20, wherein the ribs have a base end and an apex end, the method further comprising forming an end-cap at the base end and the apex end.

22. The method of claim 20, wherein the cardiac harness is formed into a tapered cylindrical configuration by rolling the silicone rubber sheets into the tapered cylindrical configuration and molding together longitudinal ends of the sheets.

23. A method of making a cardiac harness, comprising
providing a mold for receiving a plurality of longitudinally spaced ribs;
placing the ribs in the mold so that the ribs are spaced apart;
the mold having a plurality of channels between the ribs, the channels at a base end of the mold having a curve with a greater overall path length, and the channels moving toward an apex portion of the mold having a curve with a progressively shorter overall path length; and
injection molding an elastomer into the mold so that the longitudinal ribs are encased in the elastomer and curved connectors are formed in the curved channels thereby connecting adjacent ribs together.

24. The method of claim 23, wherein preformed connectors are attached to the ribs.

25. The method of claim 23, wherein as the cardiac harness is removed from the mold, the curved connectors are straightened so that the longitudinally spaced ribs have a tapered configuration, the ribs being spaced farther apart at a base end and gradually tapering to a narrower spacing between adjacent ribs at an apex end.

26. The method of claim 23, wherein the ribs have any of an undulating configuration, straight configuration, or a combination of undulating and straight configuration, in the mold.

27. The method of claim 23, wherein the connectors are flexible but substantially non-extendable.

28. The method of claim 23, wherein prior to placing the longitudinal ribs in the mold, the longitudinal ribs are jacketed in a dielectric polymer tubing.
